(19) United States
(12) Patent Application Publication
(10) Pub. No.: US 2008/0214774 A1
(43) Pub. Date: Sep. 4, 2008

(54) Thiourethane Compositions and Processes for Making and Using Same

(75) Inventors: Chad W. Brown, Bartlesville, OK (US); Jim D. Byers, Bartlesville, OK (US); Mitchell D. Refvik, Bartlesville, OK (US)

Correspondence Address: CHEVRON PHILLIPS CHEMICAL COMPANY LP, LAW DEPARTMENT - IP, P.O BOX 4910, THE WOODLANDS, TX, 77387-4910, US

(73) Assignee: Chevron Phillips Chemical Company, LP

(21) Appl. No.: 11/680,842

(22) Filed: Mar. 1, 2007

(51) Int. Cl. C08G 63/00; C07C 231/00

(52) U.S. Cl. 528/283; 528/290; 564/124

(57) ABSTRACT

Thiourethane prepolymer compositions, thiourethane polymer compositions, methods of making the compositions, and methods of using the thiourethane polymer compositions are provided. The thiourethane polymer composition can be produced by contacting a thiol ester composition and an isocyanate composition to produce a prepolymer composition and then curing the prepolymer composition to produce the thiourethane polymer composition. The prepolymer composition can also include a property modifying agent. In some embodiments, the thiol ester compositions include thiol esters, hydroxy thiol esters, and cross-linked thiol esters.

### BACKGROUND OF THE INVENTION

1. Field of the Invention

The invention relates to thiourethane compositions generally made from a reaction of thiol ester compositions and an isocyanate. The invention also relates to the processes for preparing such compositions and uses for the compositions.

2. Description of Related Art

The chemical industry strives to make products, such as prepolymers, adhesives, sealants, fertilizers, coatings, foams, and fuels, with less expensive feedstocks that are in abundant supply. As the fossil fuels slowly deplete over time, alternative sources are always being sought as replacements for fuels. Additionally, the chemical industry continuously strives to produce products and use feedstocks that are environmentally friendly in order to reduce potential hazards and risks related to safety and environmental issues.

### SUMMARY OF THE INVENTION

The present invention provides a prepolymer composition that is produced by reacting a thiol ester composition with an isocyanate composition. In embodiments, the thiol ester composition can be a hydroxy thiol ester composition; alternatively, a crosslinked thiol ester composition; alternatively, a mercaptanized unsaturated ester composition; alternatively, a mercaptanized epoxidized ester composition; or alternatively, a crosslinked mercaptanized unsaturated ester composition. The isocyanate compositions described herein can be used to prepare the polymer composition. In an aspect, the prepolymer composition can include a property modifying agent. A solvent can also be used in producing the prepolymer composition.

The prepolymer composition can be cured to produce a polymer composition that has useful properties in such applications as sealants and adhesives. The curing can occur by exposing the prepolymer composition to moisture. Other suitable means of curing can also be used.

In addition to the prepolymer and polymer compositions, methods of making the compositions are also provided as embodiments of the present invention. In an embodiment, the thiol ester composition is contacted with the isocyanate composition to form a prepolymer composition. The prepolymer composition is then cured to produce the polymer. In some embodiments, a catalyst is used to produce the prepolymer composition. In another aspect, a property modifying agent is used to produce the prepolymer composition.

The prepolymer composition can be used in one-part moisture-cure applications. In another aspect, the prepolymer composition can be used in multi-component cure systems. In multi-component cure systems, the thiourethane prepolymer composition containing unreacted, excess isocyanate is kept separate from other isocyanate reactive components to prevent the isocyanate from reacting with the other components. When the multi-component cure system is in use, the components are either mixed together a short period of time prior to being applied or the components are applied separately and mix shortly thereafter by contacting each other. When the components are mixed together prior to application, a static mixer or the like can be used. The catalyst in multi-component cure systems can be a separate, second catalyst or it can be residual catalyst that was used in forming the thiourethane prepolymer composition. Other components suitable for use in multi-component cure systems, such as fillers, will be apparent to those of skill in the art and are to be considered within the scope of the present invention. Once the components are applied, they are then cured to produce the thiourethane polymer composition. In an embodiment, the multi-component cure system can use a polyol and optionally a catalyst to cure the prepolymer composition.

### DETAILED DESCRIPTION OF THE INVENTION

In this specification “natural” refers to materials obtained, by any method, from naturally occurring fruits, nuts, vegetables, plants, and animals. As an example, natural source oil refers to source oils extracted, and optionally purified, from naturally occurring fruits, nuts, vegetables, plants, and animals. Additionally, unsaturated natural source oil refers to unsaturated source oils extracted, and optionally purified, from naturally occurring fruits, nuts, vegetables, plants, and animals. As another example, the unsaturated natural source oil can be derived from genetically modified nuts, vegetables, plant, and animal sources. As yet another example, the unsaturated natural source oil comprises a triglyceride derived from genetically modified nuts, vegetables, plant, and animal sources.

In this specification, “natural source raw material” refers to materials obtained by extraction, chemical breakdown, or chemical processing of “natural” materials. A non-limiting example includes natural source oils that can be extracted from naturally occurring fruits, nuts, vegetables, plants and animals. As another non-limiting example, glycerol and carboxylic acids or carboxylic acid esters, saturated or unsaturated, can be produced and isolated by the chemical processing of triglycerides extracted from naturally occurring fruits, nuts, vegetables, plants, and animals.

In this specification “synthetic” refers to materials produced from chemical building blocks not directly derived from natural sources. For example, synthetic unsaturated ester oil can be produced by the reaction of synthetic ethylene glycol and a synthetic carboxylic acid, e.g. acrylic acid or propionic acid. Other types of synthetic materials will be apparent to those of skill in the art and are to be considered within the scope of the present invention.

Regardless of the definitions of natural and synthetic, the materials described herein can be produced from a combination of natural and synthetic materials, which can be referred to as “semi-synthetic.” As a non-limiting example, the unsaturated ester oils described in this specification can be obtained or produced from a combination of synthetic and natural source raw materials. For example, the unsaturated ester oil can be produced by the reaction of synthetic ethylene glycol and oleic acid isolated from a natural source oil. Alternatively, the unsaturated ester oil can be produced from the reaction of glycerol isolated from natural source oils and a synthetic carboxylic acid, e.g. acrylic acid. Alternatively, the unsaturated ester oil can be produced from glycerol and oleic acid isolated from natural source oils.

In this specification, “thiol ester composition” refers to an ester composition that includes “thiol ester molecules.” The thiol ester molecule has at least one thiol group and at least one ester group within the thiol ester molecule.

In this specification, “hydroxy thiol ester composition” refers to an ester composition that includes “hydroxy thiol ester molecules.” The hydroxy thiol ester molecule has at least one thiol group, at least one ester group, and at least one hydroxy or alcohol group within the hydroxy thiol ester molecule. Alternatively, the alcohol group and the thiol group can be combined in the same group, which can be referred to as an “α-hydroxy thiol group.”

In this specification, “unsaturated ester composition” refers to an ester composition that includes unsaturated ester molecules. The unsaturated ester molecules have at least one ester group and at least one carbon-carbon double bond within the unsaturated ester molecule.

In this specification, “epoxidized unsaturated ester composition” refers to an ester composition that has been produced by epoxidizing an unsaturated ester composition.

In this specification, “thiourethane” refers to a urethane composition that includes molecules having at least one of the following structure:

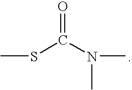

The presence of the thiourethane group can be determined by methods known to those skilled in the art, such as infrared spectroscopy, Raman spectroscopy, ¹³C NMR, and the like.

### Thiourethane Prepolymer Compositions

In an aspect, the prepolymer composition of the present invention can be described as a reaction product of a thiol ester composition, an isocyanate composition, and a catalyst. In embodiments, the prepolymer composition can be called a thiourethane prepolymer composition. The thiourethane prepolymer composition can be cured. Once the thiourethane prepolymer composition has been cured, the composition can then be called a thiourethane polymer composition. Generally, the thiourethane prepolymer composition of the present invention comprises thiourethane prepolymer molecules having multiple thiourethane groups having structure G2:

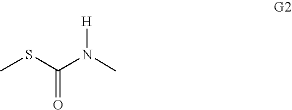

where the undesignated valencies represent the remainder of the structure of the prepolymer molecules including additional groups having structure G2. The presence of the thiourethane group G2 can be determined using techniques known to those skilled in the art, such as infrared spectroscopy, Raman spectroscopy, ¹³C NMR, and the like.

The thiourethane prepolymer composition of the present invention can be described as comprising thiourethane prepolymer molecules having multiple units D2:

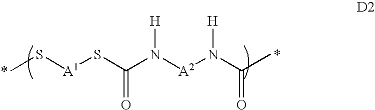

where the undesignated valencies represent the remainder of the structure of the thiourethane prepolymer molecule(s) including additional repeating units D2. In embodiments, the backbone of the thiourethane prepolymer molecule(s) having repeating unit D2 is linear; or alternatively, the backbone of the thiourethane prepolymer molecule(s) having repeating unit D2 is crosslinked. When the backbone of the thiourethane prepolymer molecule(s) having repeating unit D2 is crosslinked, A¹ and/or A² further comprise additional repeating units D2. The repeating unit D2 of the thiourethane prepolymer molecule is comprised of two different units: U1 and U2.

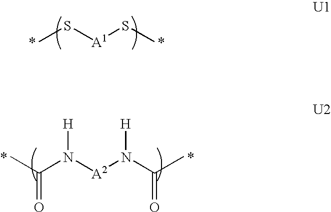

Generally, unit U1 of the thiourethane prepolymer molecule is derived from a thiol ester of the thiol ester composition and unit U2 of the thiourethane prepolymer molecule is derived from an isocyanate of the isocyanate composition. Thus, A¹ represents the remainder of the thiol ester molecule (including ester groups, any other groups present in the thiol ester molecule, and optionally additional repeating units D2), and A² represents the remainder of the isocyanate molecule (including any other groups present in the isocyanate molecule and optionally additional repeating units D2). When the thiourethane prepolymer is crosslinked, either U1 (via A¹), U2 (via A²), or both can contain additional thiourethane groups. Because units U1 and U2 are derived from two different materials, the structures of these units are independent of each other. Therefore, the thiourethane prepolymer molecule(s) having the repeating unit D2 can be comprised of any combination of units U1 and U2. Thus, the thiourethane prepolymer molecule(s) having the repeating unit D2 can be described as the reaction product of a thiol ester composition, an isocyanate composition, and a catalyst, where unit U1 can be derived from any thiol ester described herein and unit U2 can be derived from any isocyanate described herein.

The thiourethane prepolymer composition can alternatively be described as a reaction product of a thiol ester composition, an isocyanate composition, and a catalyst. The thiol ester composition, the isocyanate, and the catalyst are independent elements of the thiourethane prepolymer. Therefore, the thiourethane prepolymer composition can be described as a thiourethane prepolymer composition product of any combination of the thiol ester compositions, the isocyanate compositions, and the catalysts described herein. In aspects, the thiourethane prepolymer composition can comprise linear thiourethane prepolymer molecules. In other aspects, the thiourethane prepolymer composition can comprise crosslinked thiourethane prepolymer molecules. When the thiourethane prepolymer composition comprises crosslinked thiourethane prepolymer molecules, either the thiol ester composition comprises thiol ester molecules having greater than 2 thiol groups per thiol ester molecule or the isocyanate composition comprises isocyanate molecules having greater than 2 isocyanate groups per isocyanate molecule. Alternatively, when the thiourethane prepolymer composition comprises crosslinked thiourethane prepolymer molecules, the thiol ester composition comprises thiol ester molecules having greater than 2 thiol groups per thiol ester molecule and the isocyanate composition comprises isocyanate molecules having at least 2 isocyanate groups per isocyanate molecule.

Generally, the thiol ester composition comprises thiol ester molecules having at least 2 thiol groups and the isocyanate composition comprises isocyanate molecules having at least 2 isocyanate groups. Additional embodiments regarding the number or average number of thiol groups present in the thiol ester molecules of the thiol ester composition are described herein and are generally applicable to the description of the thiourethane prepolymer composition as a reaction product of a thiol ester composition, an isocyanate composition, and a catalyst. Additional embodiments regarding the number or average number of isocyanate groups present in the isocyanate molecules of the isocyanate composition are described herein and are generally applicable to the description of the thiourethane prepolymer composition as a reaction product of a thiol ester composition, an isocyanate composition, and a catalyst.

In embodiments, the thiol ester composition utilized to produce the thiourethane prepolymer composition can comprise a hydroxy thiol ester; alternatively, a crosslinked thiol ester; alternatively, a mercaptanized unsaturated ester; alternatively, a mercaptanized epoxidized ester; or alternatively, a crosslinked mercaptanized unsaturated ester. In non-limiting embodiments, the thiol ester composition can comprise a mercaptanized natural source oil; alternatively, mercaptanized epoxidized natural source oil; alternatively, crosslinked mercaptanized natural source oil; or alternatively, crosslinked mercaptanized epoxidized natural source oil. In further non-limiting embodiments, the thiol ester composition comprises mercaptanized soybean oil; alternatively, a mercaptanized castor oil; alternatively, a mercaptanized epoxidized soybean oil; or alternatively, a crosslinked mercaptanized soybean oil. Other thiol esters are described herein and can generally be utilized in the thiol ester compositions to describe the thiourethane prepolymer composition that is a reaction product of a thiol ester composition, an isocyanate composition, and a catalyst. Additionally, other aspects of the thiol ester materials (e.g. average number of thiol groups per thiol ester molecule, thiol sulfur content, etc.) are described herein and can be utilized to further describe the thiol esters of the thiol ester compositions. Besides the thiol ester compositions described herein, other suitable thiol ester compositions will be apparent to those persons having ordinary skill in the art, can be used, and are to be considered within the scope of the present invention.

Generally, the isocyanate composition can comprise, singly or in any combination, any isocyanate described herein. In embodiments, the isocyanate composition can comprise aliphatic isocyanates, cycloaliphatic isocyanates, aromatic isocyanates, or mixtures thereof. In some embodiments, the isocyanate composition can comprise an aliphatic isocyanate; alternatively, a cycloaliphatic isocyanate; or alternatively, an aromatic isocyanate composition. Particular isocyanates having at least two isocyanate groups are described herein and can generally be utilized in the isocyanate compositions describing the thiourethane prepolymer composition as a reaction product of a catalyst, a thiol ester composition, and an isocyanate composition. Additionally, other aspects of the isocyanate materials (e.g. number or average number of isocyanate groups per isocyanate molecule, etc.) are described herein and can be utilized to further describe the isocyanate composition.

Generally, the catalyst can comprise, singly or in any combination, any catalyst described herein. In some embodiments, the catalyst can be a tin catalyst, an amine catalyst, a bismuth catalyst, an iron catalyst, or combinations thereof. In embodiments, the catalyst can be selected from the group consisting of a tertiary amine, an organo-tin compound, an amine initiated polypropylene glycol, and combinations thereof. In some embodiments the catalyst can be an amine. In other embodiments, the catalyst can be a tin compound. In some embodiments, the catalyst is a tertiary amine. In other embodiments the catalyst can be aliphatic amine; or alternatively, an aromatic amine. In other embodiments, the catalyst can be a polyether amine; alternatively, a polyalkylene amine; or alternatively, a tertiary amine polyol. In yet other embodiments, the amine catalyst can be a polyamine comprising at least two amine groups. In some amine catalyst embodiments, the catalyst can be 1,8-diazabicyclo[5,4,0]undec-7-ene [DBU-CAS# 6674-22-2]; alternatively, 1,4-diazabicyclo[2.2.2]octane [DABCO-CAS# 280-57-9]); or alternatively, triethylamine. In a tin compound catalyst embodiment, the tin compound can be dibutyl tin dilaurate. Other suitable catalysts will be apparent to those of skill in the art and are to be considered within the scope of the present invention.

Generally, the thiol ester composition, the isocyanate composition, and the catalyst are independent elements of the thiourethane prepolymer composition described as the reaction product of a thiol ester composition, an isocyanate composition, and a catalyst. Therefore, the thiourethane prepolymer composition can be described as the reaction product of any combination of the thiol ester composition, the isocyanate composition, and the catalyst described herein. In embodiments, the thiourethane prepolymer composition can be described as the reaction product of a catalyst and a thiol ester composition with an isocyanate composition comprising an isocyanate having at least two isocyanate groups. In embodiments, the thiourethane prepolymer composition can be described as the reaction product of a catalyst, a thiol ester composition, and an isocyanate composition comprising an aliphatic isocyanate having at least two isocyanate groups, a cycloaliphatic isocyanate having at least two isocyanate groups, an aromatic isocyanate having at least two isocyanate groups, or mixtures thereof. In some embodiments, the thiourethane prepolymer composition can be described as the reaction product of a catalyst, a thiol ester composition, and an isocyanate composition comprising an aliphatic isocyanate having at least two isocyanate groups; alternatively, a cycloaliphatic isocyanate having at least two isocyanate groups; or alternatively, an aromatic isocyanate having at least two isocyanate groups. Particular aliphatic, cycloaliphatic, and aromatic isocyanates having at least two isocyanate groups are described herein and can generally be utilized in the isocyanate compositions describing the thiourethane prepolymer composition as a reaction product of a thiol ester composition, an isocyanate composition, and a catalyst. Additionally, other aspects of the isocyanate materials (e.g. number or average number of isocyanate groups per isocyanate molecule, etc.) are described herein and can be utilized to further describe the isocyanate composition utilized in describing the thiourethane prepolymer composition as a reaction product of a thiol ester composition, an isocyanate composition, and a catalyst.

In embodiments, the thiourethane prepolymer composition can be described as the reaction product of a catalyst, a hydroxy thiol ester composition, and an isocyanate composition comprising an aliphatic isocyanate having at least two isocyanate groups, a cycloaliphatic isocyanate having at least two isocyanate groups, an aromatic isocyanate having at least two isocyanate groups, or mixtures thereof. In some embodiments, the thiourethane prepolymer composition can be described as the reaction product of a catalyst, a hydroxy thiol ester composition, and an isocyanate composition comprising an aliphatic isocyanate having at least two isocyanate groups; alternatively, a cycloaliphatic isocyanate having at least two isocyanate groups; or alternatively, an aromatic isocyanate having at least two isocyanate groups. Particular aliphatic, cycloaliphatic, and aromatic isocyanates having at least two isocyanate groups are described herein and can generally be utilized in the isocyanate compositions describing the thiourethane prepolymer composition as a reaction product of a catalyst, a hydroxy thiol ester composition, and an isocyanate composition. Additionally, other aspects of the isocyanate materials (e.g. number or average number of isocyanate groups per isocyanate molecule, etc.) are described herein and can be utilized to further describe the isocyanate composition utilized in describing the thiourethane prepolymer composition as a reaction product of a hydroxy thiol ester composition, an isocyanate composition, and a catalyst.

In embodiments, the thiourethane prepolymer composition can be described as the reaction product of a catalyst, a crosslinked thiol ester composition, and an isocyanate composition comprising an aliphatic isocyanate having at least two isocyanate groups, a cycloaliphatic isocyanate having at least two isocyanate groups, an aromatic isocyanate having at least two isocyanate groups, or mixtures thereof. In some embodiments, the thiourethane prepolymer composition can be described as the reaction product of a catalyst, a crosslinked thiol ester composition, and an isocyanate composition comprising an aliphatic isocyanate having at least two isocyanate groups; alternatively, a cycloaliphatic isocyanate having at least two isocyanate groups; or alternatively, an aromatic isocyanate having at least two isocyanate groups. Particular aliphatic, cycloaliphatic, and aromatic isocyanates having at least two isocyanate groups are described herein and can generally be utilized in the isocyanate compositions describing the thiourethane prepolymer composition as a reaction product of a catalyst, a crosslinked thiol ester composition, and an isocyanate composition. Additionally, other aspects of the isocyanate materials (e.g. number or average number of isocyanate groups per isocyanate molecule, etc.) are described herein and can be utilized to further describe the isocyanate composition utilized in describing the thiourethane prepolymer composition as a reaction product of a crosslinked thiol ester composition, an isocyanate composition, and a catalyst.

In embodiments, the thiourethane prepolymer composition can be described as the reaction product of a catalyst, a thiol ester composition comprising a mercaptanized unsaturated ester, and an isocyanate composition comprising an aliphatic isocyanate having at least two isocyanate groups, a cycloaliphatic isocyanate having at least two isocyanate groups, an aromatic isocyanate having at least two isocyanate groups, or mixtures thereof. In some embodiments, the thiourethane prepolymer composition can be described as the reaction product of a catalyst, a thiol ester composition comprising a mercaptanized unsaturated ester, and an isocyanate composition comprising an aliphatic isocyanate having at least two isocyanate groups; alternatively, a cycloaliphatic isocyanate having at least two isocyanate groups; or alternatively, an aromatic isocyanate having at least two isocyanate groups. Particular aliphatic, cycloaliphatic, and aromatic isocyanates having at least two isocyanate groups are described herein and can generally be utilized in the isocyanate compositions describing the thiourethane prepolymer composition as a reaction product of a catalyst, a thiol ester composition comprising a mercaptanized unsaturated ester, and an isocyanate composition. Additionally, other aspects of the isocyanate materials (e.g. number or average number of isocyanate groups per isocyanate molecule, etc.) are described herein and can be utilized to further describe the isocyanate composition utilized in describing the thiourethane prepolymer composition as a reaction product of a catalyst, a thiol ester composition comprising a mercaptanized unsaturated ester, and an isocyanate composition.

In embodiments, the thiourethane prepolymer composition can be described as the reaction product of a catalyst, a thiol ester composition comprising a mercaptanized epoxidized ester, and an isocyanate composition comprising an aliphatic isocyanate having at least two isocyanate groups, a cycloaliphatic isocyanate having at least two isocyanate groups, an aromatic isocyanate having at least two isocyanate groups, or mixtures thereof. In some embodiments, the thiourethane prepolymer composition can be described as the reaction product of a catalyst, a thiol ester composition comprising a mercaptanized epoxidized ester, and an isocyanate composition comprising an aliphatic isocyanate having at least two isocyanate groups; alternatively, a cycloaliphatic isocyanate having at least two isocyanate groups; or alternatively, an aromatic isocyanate having at least two isocyanate groups. Particular aliphatic, cycloaliphatic, and aromatic isocyanates having at least two isocyanate groups are described herein and can generally be utilized in the isocyanate compositions describing the thiourethane prepolymer composition as a reaction product of a catalyst, a thiol ester composition comprising a mercaptanized epoxidized ester, and an isocyanate composition. Additionally, other aspects of the isocyanate materials (e.g. number or average number of isocyanate groups per isocyanate molecule, etc.) are described herein and can be utilized to further describe the isocyanate composition utilized in describing the thiourethane prepolymer composition as a reaction product of a catalyst, a thiol ester composition comprising a mercaptanized epoxidized ester, and an isocyanate composition.

In embodiments, the thiourethane prepolymer composition can be described as the reaction product of a catalyst, a thiol ester composition comprising a crosslinked mercaptanized unsaturated ester, and an isocyanate composition comprising an aliphatic isocyanate having at least two isocyanate groups, a cycloaliphatic isocyanate having at least two isocyanate groups, an aromatic isocyanate having at least two isocyanate groups, or mixtures thereof. In some embodiments, the thiourethane prepolymer composition can be described as the reaction product of a catalyst, a thiol ester composition comprising a crosslinked mercaptanized unsaturated ester, and an isocyanate composition comprising an aliphatic isocyanate having at least two isocyanate groups; alternatively, a cycloaliphatic isocyanate having at least two isocyanate groups; or alternatively, an aromatic isocyanate having at least two isocyanate groups. Particular aliphatic, cycloaliphatic, and aromatic isocyanates having at least two isocyanate groups are described herein and can generally be utilized in the isocyanate compositions describing the thiourethane prepolymer composition as a reaction product of a thiol ester composition comprising a crosslinked mercaptanized unsaturated ester, an isocyanate composition, and a catalyst. Additionally, other aspects of the isocyanate materials (e.g. number or average number of isocyanate groups per isocyanate molecule, etc.) are described herein and can be utilized to further describe the isocyanate composition utilized in describing the thiourethane prepolymer composition as a reaction product of a catalyst, a thiol ester composition comprising a crosslinked mercaptanized unsaturated ester, and an isocyanate composition.

In embodiments, the thiourethane prepolymer composition can be described as the reaction product of a catalyst, a thiol ester composition comprising mercaptanized natural source oil, and an isocyanate composition comprising an aliphatic isocyanate having at least two isocyanate groups, a cycloaliphatic isocyanate having at least two isocyanate groups, an aromatic isocyanate having at least two isocyanate groups, or mixtures thereof. In some embodiments, the thiourethane prepolymer composition can be described as the reaction product of a catalyst, a thiol ester composition comprising a mercaptanized natural source oil, and an isocyanate composition comprising an aliphatic isocyanate having at least two isocyanate groups; alternatively, a cycloaliphatic isocyanate having at least two isocyanate groups; or alternatively, an aromatic isocyanate having at least two isocyanate groups. Particular aliphatic, cycloaliphatic, and aromatic isocyanates having at least two isocyanate groups are described herein and can generally be utilized in the isocyanate compositions describing the thiourethane prepolymer composition as a reaction product of a catalyst, a thiol ester composition comprising a mercaptanized natural source oil, and an isocyanate composition. Additionally, other aspects of the isocyanate materials (e.g. number or average number of isocyanate groups per isocyanate molecule, etc.) are described herein and can be utilized to further describe the isocyanate composition utilized in describing the thiourethane prepolymer composition as a reaction product of a thiol ester composition comprising a mercaptanized natural source oil, an isocyanate composition, and a catalyst.

In embodiments, the thiourethane prepolymer composition can be described as the reaction product of a catalyst, a thiol ester composition comprising a mercaptanized epoxidized natural source oil, and an isocyanate composition comprising an aliphatic isocyanate having at least two isocyanate groups, a cycloaliphatic isocyanate having at least two isocyanate groups, an aromatic isocyanate having at least two isocyanate groups, or mixtures thereof. In some embodiments, the thiourethane prepolymer composition can be described as the reaction product of a catalyst, a thiol ester composition comprising a mercaptanized epoxidized natural source oil, and an isocyanate composition comprising an aliphatic isocyanate having at least two isocyanate groups; alternatively, a cycloaliphatic isocyanate having at least two isocyanate groups; or alternatively, an aromatic isocyanate having at least two isocyanate groups. Particular aliphatic, cycloaliphatic, and aromatic isocyanates having at least two isocyanate groups are described herein and can generally be utilized in the isocyanate compositions describing the thiourethane prepolymer composition as a reaction product of a catalyst, a thiol ester composition comprising a mercaptanized epoxidized natural source oil, and an isocyanate composition. Additionally, other aspects of the isocyanate materials (e.g. number or average number of isocyanate groups per isocyanate molecule, etc.) are described herein and can be utilized to further describe the isocyanate composition utilized in describing the thiourethane prepolymer composition as a reaction product of a catalyst, a thiol ester composition comprising a mercaptanized epoxidized natural source oil, and an isocyanate composition.

In embodiments, the thiourethane prepolymer composition can be described as the reaction product of a catalyst, a thiol ester composition comprising a crosslinked mercaptanized natural source oil, and an isocyanate composition comprising an aliphatic isocyanate having at least two isocyanate groups, a cycloaliphatic isocyanate having at least two isocyanate groups, an aromatic isocyanate having at least two isocyanate groups, or mixtures thereof. In some embodiments, the thiourethane prepolymer composition can be described as the reaction product of a catalyst, a thiol ester composition comprising a crosslinked mercaptanized natural source oil, and an isocyanate composition comprising an aliphatic isocyanate having at least two isocyanate groups; alternatively, a cycloaliphatic isocyanate having at least two isocyanate groups; or alternatively, an aromatic isocyanate having at least two isocyanate groups. Particular aliphatic, cycloaliphatic, and aromatic isocyanates having at least two isocyanate groups are described herein and can generally be utilized in the isocyanate compositions describing the thiourethane prepolymer composition as a reaction product of a catalyst, a thiol ester composition comprising a crosslinked mercaptanized natural source oil, and an isocyanate composition. Additionally, other aspects of the isocyanate materials (e.g. number or average number of isocyanate groups per isocyanate molecule, etc.) are described herein and can be utilized to further describe the isocyanate composition utilized in describing the thiourethane prepolymer composition as a reaction product of a catalyst, a thiol ester composition comprising a crosslinked mercaptanized natural source oil, and an isocyanate composition.

In embodiments, the thiourethane prepolymer composition can be described as the reaction product of a catalyst, a thiol ester composition comprising a mercaptanized soybean oil, and an isocyanate composition comprising an aliphatic isocyanate having at least two isocyanate groups, a cycloaliphatic isocyanate having at least two isocyanate groups, an aromatic isocyanate having at least two isocyanate groups, or mixtures thereof. In some embodiments, the thiourethane prepolymer composition can be described as the reaction product of a catalyst, a thiol ester composition comprising a mercaptanized soybean oil, and an isocyanate composition comprising an aliphatic isocyanate having at least two isocyanate groups; alternatively, a cycloaliphatic isocyanate having at least two isocyanate groups; or alternatively, an aromatic isocyanate having at least two isocyanate groups. Particular aliphatic, cycloaliphatic, and aromatic isocyanates having at least two isocyanate groups are described herein and can generally be utilized in the isocyanate compositions describing the thiourethane prepolymer composition as a reaction product of a catalyst, a thiol ester composition comprising a mercaptanized soybean oil, and an isocyanate composition. Additionally, other aspects of the isocyanate materials (e.g. number or average number of isocyanate groups per isocyanate molecule, etc.) are described herein and can be utilized to further describe the isocyanate composition utilized in describing the thiourethane prepolymer composition as a reaction product of a catalyst, a thiol ester composition comprising a mercaptanized soybean oil, and an isocyanate composition.

In embodiments, the thiourethane prepolymer composition can be described as the reaction product of a catalyst, a thiol ester composition comprising a mercaptanized castor oil, and an isocyanate composition comprising an aliphatic isocyanate having at least two isocyanate groups, a cycloaliphatic isocyanate having at least two isocyanate groups, an aromatic isocyanate having at least two isocyanate groups, or mixtures thereof. In some embodiments, the thiourethane prepolymer composition can be described as the reaction product of a catalyst, a thiol ester composition comprising a mercaptanized castor oil, and an isocyanate composition comprising an aliphatic isocyanate having at least two isocyanate groups; alternatively, a cycloaliphatic isocyanate having at least two isocyanate groups; or alternatively, an aromatic isocyanate having at least two isocyanate groups. Particular aliphatic, cycloaliphatic, and aromatic isocyanates having at least two isocyanate groups are described herein and can generally be utilized in the isocyanate compositions describing the thiourethane prepolymer composition as a reaction product of a catalyst, a thiol ester composition comprising a mercaptanized castor oil, and an isocyanate composition. Additionally, other aspects of the isocyanate materials (e.g. number or average number of isocyanate groups per isocyanate molecule, etc.) are described herein and can be utilized to further describe the isocyanate composition utilized in describing the thiourethane prepolymer composition as a reaction product of a catalyst, a thiol ester composition comprising a mercaptanized castor oil, and an isocyanate composition.

In embodiments, the thiourethane prepolymer composition can be described as the reaction product of a catalyst, a thiol ester composition comprising a mercaptanized epoxidized soybean oil, and an isocyanate composition comprising an aliphatic isocyanate having at least two isocyanate groups, a cycloaliphatic isocyanate having at least two isocyanate groups, an aromatic isocyanate having at least two isocyanate groups, or mixtures thereof. In some embodiments, the thiourethane prepolymer composition can be described as the reaction product of a catalyst, a thiol ester composition comprising a mercaptanized epoxidized soybean oil, and an isocyanate composition comprising an aliphatic isocyanate having at least two isocyanate groups; alternatively, a cycloaliphatic isocyanate having at least two isocyanate groups; or alternatively, an aromatic isocyanate having at least two isocyanate groups. Particular aliphatic, cycloaliphatic, and aromatic isocyanates having at least two isocyanate groups are described herein and can generally be utilized in the isocyanate compositions describing the thiourethane prepolymer composition as a reaction product of a catalyst, a thiol ester composition comprising a mercaptanized epoxidized soybean oil, and an isocyanate composition. Additionally, other aspects of the isocyanate materials (e.g. number or average number of isocyanate groups per isocyanate molecule, etc.) are described herein and can be utilized to further describe the isocyanate composition utilized in describing the thiourethane prepolymer composition as a reaction product of a catalyst, a thiol ester composition comprising a mercaptanized epoxidized soybean oil, and an isocyanate composition.

In embodiments, the thiourethane prepolymer composition can be described as the reaction product of a catalyst, a thiol ester composition comprising a crosslinked mercaptanized soybean oil, and an isocyanate composition comprising an aliphatic isocyanate having at least two isocyanate groups, a cycloaliphatic isocyanate having at least two isocyanate groups, an aromatic isocyanate having at least two isocyanate groups, or mixtures thereof. In some embodiments, the thiourethane prepolymer composition can be described as the reaction product of a catalyst, a thiol ester composition comprising a crosslinked mercaptanized soybean oil, and an isocyanate composition comprising an aliphatic isocyanate having at least two isocyanate groups; alternatively, a cycloaliphatic isocyanate having at least two isocyanate groups; or alternatively, an aromatic isocyanate having at least two isocyanate groups. Particular aliphatic, cycloaliphatic, and aromatic isocyanates having at least two isocyanate groups are described herein and can generally be utilized in the isocyanate compositions describing the thiourethane prepolymer composition as a reaction product of a catalyst, a thiol ester composition comprising a crosslinked mercaptanized soybean oil, and an isocyanate composition. Additionally, other aspects of the isocyanate materials (e.g. number or average number of isocyanate groups per isocyanate molecule, etc.) are described herein and can be utilized to further describe the isocyanate composition utilized in describing the thiourethane prepolymer composition as a reaction product of a catalyst, a thiol ester composition comprising a crosslinked mercaptanized soybean oil, and an isocyanate composition.

In embodiments, the thiourethane prepolymers of the present invention can be described as a product produced by any process described herein capable of producing the thiourethane prepolymer composition and can be further described as being produced using any embodiments of the processes described herein.

In embodiments, the thiourethane prepolymer composition of the present invention can be further described by its properties. In some embodiments, the thiourethane prepolymer composition described as the reaction product of a thiol ester composition, an isocyanate composition, and a catalyst can have an apparent viscosity ranging from 2500 centipoises (cP) to 10,000 cP at a temperature of 120° F. (49° C.). In other embodiments, the thiourethane prepolymer composition of the present invention has an apparent viscosity ranging from 3,000 cP to 9,000 cP at 120° F. (49° C.); alternatively, ranging from 3,500 cP to 8,000 cP at 120° F. (49° C.); alternatively, ranging from 4,000 cP to 7,000 cP at 120° F. (49° C.); or alternatively, ranging from 4,500 cP to 6,000 cP at 120° F. (49° C.). The apparent viscosities can be measured using a Brookfield® viscometer.

The properties of the thiourethane prepolymer composition can be adjusted by adding other components to the composition. For example, a solvent can be added to the thiourethane prepolymer composition during synthesis or afterwards. The solvent can be useful in adjusting the viscosity of the thiourethane prepolymer composition. Some solvents can lower the viscosity of the thiourethane prepolymer composition to enable the composition to be applied more easily.

In an embodiment, the solvent can be a hydrocarbon solvent, a halogenated hydrocarbon solvent, a ketone solvent, a carbonate solvent, an ester solvent, an ether solvent, or any combination thereof. In some embodiments, the solvent can be a hydrocarbon solvent; alternatively, a halogenated hydrocarbon solvent; alternatively, a ketone solvent; alternatively, a carbonate solvent; alternatively, an ester solvent; or alternatively, an ether solvent.

In an embodiment, the solvent can comprise, or alternatively consist essentially of, a C₄ to C₂₀ saturated hydrocarbon; alternatively, a C₄ to C₁₀ saturated hydrocarbon. In some embodiments, the solvent can comprise, or alternatively consist essentially of, a C₆ to C₂₀ aromatic hydrocarbon; or alternatively, C₆ to C₂₀ aromatic hydrocarbon. In an embodiment, the solvent can comprise, or alternatively consist essentially of, a C₁ to C₁₅ halogenated hydrocarbon; alternatively, a C₁ to C₁₀ halogenated hydrocarbon; or alternatively, C₁ to C₅ halogenated hydrocarbon. In some embodiments, the solvent can comprise, or alternatively consist essentially of, a C₁ to C₁₀ ketone; or alternatively, a C₁ to C₅ ketone. In some embodiments, the solvent can comprise, or alternatively consist essentially of, a C₁ to C₁₀ carbonate; or alternatively, a C₁ to C₅ carbonate. In some embodiments, the solvent can comprise, or alternatively consist essentially of, a C₁ to C₁₀ ester; or alternatively, a C₁ to C₁₀ ester. In some embodiments, the solvent can comprise, or alternatively consist essentially of, a C₁ to C₁₀ ether; or alternatively, a C₁ to C₁₀ ether.

Suitable saturated hydrocarbon solvents that can be utilized, either singly or in any combination, include, but are not limited to, pentane, n-hexane, hexanes, cyclopentane, cyclohexane, n-heptane, heptanes, n-octane, and petroleum distillate. Suitable aromatic hydrocarbon solvents that can be utilized, either singly or in any combination, include, but are not limited to, benzene, toluene, mixed xylenes, ortho-xylene, meta-xylene, para-xylene, and ethylbenzene. Suitable halogenated solvents that can be utilized, either singly or in any combination, include, but are not limited to, carbon tetrachloride, chloroform, methylene chloride, dichloroethane, trichloroethane, chlorobenzene, and dichlorobenzene. Suitable ketone solvents that can be utilized, either singly or in any combination, include, but are not limited to, acetone, and methyl ethyl ketone. Suitable carbonate solvents that can be utilized, either singly or in any combination, include, but are not limited to, dimethyl carbonate, diethyl carbonate, propylene carbonate, and glycerol carbonate. Suitable ester solvents that can be utilized, either singly or in any combination, include, but are not limited to, methyl acetate, ethyl acetate, and butyl acetate. Suitable ether solvents that can be utilized, either singly or in any combination, include, but are not limited to, dimethyl ether, diethyl ether, methyl ethyl ether, diethers of glycols (e.g. dimethyl glycol ether), furans, dihydrofuran, substituted dihydrofurans, tetrahydrofuran (THF), tetrahydropyrans, 1,3-dioxanes, and 1,4-dioxanes. In some embodiments, the solvent can be methyl ethyl ketone, glycerol carbonate, acetone, hexane, petroleum distillate, butyl acetate, toluene, benzene, or combinations thereof. Other suitable solvents will be apparent to those of ordinary skill in the art and are to be considered within the scope of the present invention.

In another example, the properties of the thiourethane prepolymer composition can be modified by including a property modifying agent within one of the compositions used to produce the thiourethane prepolymer composition or as a separate composition used to make the thiourethane prepolymer. Consequently, the thiourethane prepolymer composition can be described as a reaction product of a thiol ester composition, an isocyanate composition, a catalyst, and a property modifying agent composition. Applicable property modifying agents are described herein and can be used without limitation to describe the thiourethane prepolymer composition as a reaction product. In an embodiment, the property modifying agent can be a compound having an active hydrogen group. Applicable active hydrogen groups which can be present in the property modifying agents are described herein and can be used without limitation to describe the thiourethane prepolymer composition as a reaction product. Additionally, specific compounds containing active hydrogen groups are described herein and can be used without limitation to describe the thiourethane prepolymer composition as a reaction product.

The thiourethane prepolymer compositions described herein can be stored for extended periods of time before being used in curing systems without substantially adverse effects related to its properties, i.e., the properties remain substantially unchanged during the shelf life. In an aspect, the thiourethane prepolymer compositions can have a shelf life of at least six months.

The thiourethane prepolymer composition can be used in numerous applications. As an example, the thiourethane prepolymer composition can be used in one-part moisture-cure systems. In such one-part systems, moisture is typically used to cure the thiourethane prepolymer composition over a time period that can range from about 2 hours to about 24 hours. If a faster cure is desired, heat can be used to expedite curing. Other suitable curing profiles can be used as will be apparent to those of skill in the art.

As another example, the thiourethane prepolymer composition can be used in multi-component cure systems. Multi-component cure systems typically comprise additional compounds that can react with excess isocyanate in the thiourethane prepolymer composition. In an example, the thiourethane prepolymer composition can be used in a multi-component cure system comprising the thiourethane prepolymer composition, an active hydrogen agent, and optionally a second catalyst to cure the thiourethane prepolymer composition. Any of the active hydrogen agents described herein can be used in producing the cured thiourethane polymer composition. For example, in some embodiments, the active hydrogen agent is a polyol, such as castor oil. In multi-component cure systems, the thiourethane prepolymer composition containing unreacted, excess isocyanate is kept separate from other active hydrogen components to prevent the isocyanate from reacting with the other active hydrogen components. In some embodiments, the second catalyst can be residual catalyst remaining in the thiourethane prepolymer composition. In other embodiments, a different catalyst can be used. The type of catalyst and the location of the catalyst with respect to the components can be selected based upon the desired outcome. For example, some catalysts are good at catalyzing water, so the catalyst can be located in any of the components. Other catalysts are better at catalyzing alcohols. Other components suitable for use in multi-component cure systems, such as fillers, will be apparent to those of skill in the art and are to be considered within the scope of the present invention. Once the components are applied, they are then cured to produce the thiourethane polymer composition.

When the multi-component cure system is in use, the components are either mixed together a short period of time prior to being applied or the components are applied separately and mix shortly thereafter by contacting each other. When the components are mixed together prior to application, a static mixer or the like can be used.

Besides the moisture cure systems described herein, other suitable uses or applications for the thiourethane prepolymer composition will be apparent to those of skill in the art and are to be considered within the scope of the present invention.

### Thiourethane Polymer Compositions

The thiourethane prepolymer compositions described herein can be used to produce thiourethane polymer compositions. The thiourethane prepolymer compositions can be cured to produce the thiourethane polymer compositions. The components that were used to produce and describe the thiourethane prepolymer composition can also be used to produce and describe the thiourethane polymer composition. Minimally, the thiourethane polymer compositions comprise polymer molecules comprising multiple thiourethane groups having previously described structure G2. The thiourethane polymer composition of the present invention can also be described as comprising thiourethane molecules having multiple previously described units D2 composed of previously described units U1 and U2. Because the thiourethane composition is produced from the thiourethane prepolymer composition, the thiourethane polymer composition can be further described using the aspects of the thiourethane prepolymer described herein. Depending upon the particular thiourethane polymer produced from the thiourethane prepolymer composition, the thiourethane polymer molecules may not have any more thiourethane groups than the thiourethane prepolymer molecules. For example, the moisture cure of a thiourethane prepolymer composition does not substantially add any additional thiourethane groups to the polymer. However, if a multi-component system utilizes a thiol compound as an active hydrogen agent then additional thiourethane groups can be formed.

In embodiments, the thiourethane polymer composition of the present invention can be further described by its properties. For example, in some embodiments, the thiourethane polymer composition has a full cure lap shear strength on oak-to-oak substrate per ASTM D1002 in a range of about 300 psi (2068 kPa) to about 1500 psi (10340 kPa); alternatively, from 350 psi (2413 kPa) to 1400 psi (9653 kPa); alternatively, from about 400 psi (2758 kPa) to about 1300 psi (8963 kPa); alternatively, from 450 psi (3103 kPa) to 1200 psi (8274 kPa); alternatively, from 500 psi (3447 kPa) to 1100 psi (7584 kPa); alternatively, from 550 psi (3792 kPa) to 1000 psi (6895 kPa); alternatively, from 600 psi (4137 kPa) to 900 psi (6205 kPa); or alternatively, from 650 psi (4482 kPa) to 800 psi (5516 kPa). The full cure values are stated as full cure strength prior to weathering. The lap shear strength can also be stated as the strength after weathering in accordance with the methods described in ASTM D 1151-84. Generally, the lap shear strength decreases upon weathering. In an embodiment, weathering results in a less than 50 percent reduction in lap shear strength; alternatively, less than 40 percent reduction in lap shear strength; alternatively, less than 35 percent reduction in lap shear strength; alternatively, less than 30 percent reduction in lap shear strength; or alternatively, less than 25 percent reduction in lap shear strength.

Other properties can also be used to describe the thiourethane polymer composition of the present invention. In some embodiments, the thiourethane polymer composition has an elongation value that ranges from 40% to 110%; alternatively, from 50% to 100%; alternatively, from 60% to 90%; or alternatively, from 70% to 80%. Elongation was measured in accordance with ASTM D412. In an aspect, the thiourethane polymer composition has an average shore hardness A ranging from 25 A to 60 A; alternatively, from 30 A to 50 A; alternatively, from 35 A to 40 A. Shore hardness A was measured in accordance with ASTM D2240. As another example, the thiourethane polymer composition has an average modulus at 25% elongation ranging from 350 psi (2413 kPa) to 1175 psi (8101 kPa); alternatively, from 400 psi (2758 kPa) to 1100 psi (7584 kPa); alternatively, from 500 psi (3447 kPa) to 1000 psi (6895 kPa); alternatively, from 600 psi (4137 kPa) to 900 psi (6205 kPa); or alternatively, from 700 psi (4826 kPa) to 800 psi (5516 kPa). In another aspect, the thiourethane polymer composition has an average modulus at 50% elongation ranging from 250 psi (1724 kPa) to 850 psi (5861 kPa); alternatively, from 300 psi (2068 kPa) to 800 psi (5516 kPa); alternatively, from 400 psi (2758 kPa) to 700 psi (4826 kPa); or alternatively, 500 psi (3447 kPa) to 600 psi (4137 kPa). In yet another example, the thiourethane polymer composition has an average tensile strength ranging from 200 psi (1379 kPa) to 700 psi (4826 kPa); alternatively, from 300 psi (2068 kPa) to 600 psi (4137 kPa); or alternatively, 400 psi (2758 kPa) to 500 psi (3447 kPa). Tensile modulus was measured in accordance with ASTM D412. In another aspect, it is believed that the thiourethane polymer composition has a joint mobility test result that ranges from −45% to +45%; or alternatively, from −25% to +25%.

The properties of the thiourethane polymer composition can be adjusted by adding other components, such as a property modifying agent, when the thiourethane prepolymer composition is being prepared. The property modifying agent can be added to modify various properties within the thiourethane prepolymer composition and the thiourethane polymer composition. For example, material(s) can be added to improve the flexibility of the thiourethane polymer composition. In an embodiment, the property modifying agent can be a compound having an active hydrogen group. Applicable active hydrogen groups which may be present in the property modifying agents are described herein and may be used without limitation in respect to the thiourethane prepolymer and/or thiourethane polymer composition. Additionally, specific compounds containing active hydrogen groups are described herein and may be used without limitation to describe the thiourethane prepolymer and/or thiourethane polymer composition. For example, in an embodiment, a polyol, can be added as the property modifying agent to the thiourethane prepolymer composition when it is being prepared. In some embodiments, the polyol can be a polypropylene glycol. Oligomeric reagents can be used in embodiments where flexibility is needed, such as when the thiourethane polymer composition is being used as an adhesive or a sealant. In some embodiments, the oligomeric reagent can be an oligomeric polyol, polyether, polyester, polyamines, polyether esters, and combinations thereof. The property modifying agent can be used to provide other properties, such as strength and adhesion to the polymers produced in accordance with embodiments of the present invention. In an aspect, the property modifying agent includes an active hydrogen group. Suitable property modifying agents can include trifunctional oligomers, tackifiers, polybutadiene, polyether amines (such as Jeffamine® polymers), ethers, urea, di(hydroxyethyl)disulfide (DIHEDS), and the like. The property modifying agent can be added either during synthesis of the thiourethane prepolymer or added immediately preceding or during the curing of the thiourethane polymer in a multi-component system. If the property modifying agent contains an active hydrogen group and is added during synthesis, it is believed that the resulting prepolymer composition will have slightly different properties than if the property modifying agent containing an active hydrogen group is added afterwards.

In an aspect, the oligomeric property modifying agent, regardless of type, can have a Mₙ that ranges between 1000 and 10,000; alternatively, between 1100 and 9,000; alternatively, between 1200 and 8,000; alternatively, between 1300 and 7,000; alternatively, between 1400 and 6,000; alternatively, between 1500 and 5,000; alternatively, between 1600 and 4,000; alternatively, between 1700 and 3,000; or alternatively, between 1800 and 2,000. Other suitable property modifying agents will be apparent to those of skill in the art and are to be considered within the scope of the present invention.

### Process of Making the Thiourethane Polymer Compositions

In an embodiment of the present invention, the method of producing the thiourethane polymer composition comprises contacting the thiol ester composition, the isocyanate composition, and the catalyst to produce a thiourethane prepolymer composition and curing the thiourethane prepolymer composition to produce the thiourethane polymer. Any thiol ester composition, isocyanate composition, and catalyst described herein can be used to produce the thiourethane prepolymer compositions of the present invention. The thiourethane prepolymer compositions are cured to produce the thiourethane polymer compositions. In some embodiments, curing occurs by exposing the thiourethane prepolymer composition to moisture.

In an aspect, a method of making the thiourethane polymer composition of the present invention comprises contacting the thiourethane prepolymer composition, an active hydrogen agent, and optionally a second catalyst. In an embodiment, the curing occurs in the presence of the active hydrogen agent comprising at least two active hydrogen groups selected from the group consisting of an alcohol group, a thiol group, a carboxylic acid group, an amine group, and an amide group. In some embodiments, the active hydrogen agent comprises at least two active hydrogen groups selected group the group consisting of an alcohol group, a thiol group, and amine group; or alternatively selected from the group consisting of an alcohol group and a thiol group. In some embodiments, the active hydrogen agent comprises a polyol; alternatively a polythiol; or alternatively, a polycarboxylic acid. In some embodiments, the active hydrogen agent is moisture (e.g. water). In an embodiment wherein the active hydrogen agent is moisture, the moisture can be atmospheric moisture. As previously described, the method of producing the thiourethane polymer composition can be used in multi-component curing systems. For example, the active hydrogen agent and optionally the second catalyst can be stored separately from thiourethane prepolymer composition that contains excess reactive isocyanate groups. The thiourethane prepolymer composition can be applied separately or together with the active hydrogen agent and the optional second catalyst to produce the thiourethane polymer composition, once the components cure. The second catalyst can be any catalyst described herein. The second catalyst can be the same catalyst used to produce the thiourethane prepolymer composition. The second catalyst can be residual catalyst from the reaction that produced the thiourethane prepolymer composition, or can be added independently of producing the thiourethane prepolymer composition.

The order in which the components used to produce the thiourethane prepolymer composition are mixed or added does not significantly affect the properties of the resulting thiourethane prepolymer composition, nor the ultimately produced thiourethane polymer composition. For example, in some embodiments, the components are simultaneously added to a reactor. In other embodiments, the components are fed individually in any order.

The thiourethane polymer composition can be produced by forming and then curing a thiourethane prepolymer composition. The thiourethane prepolymer composition can be any thiourethane prepolymer composition described herein. Alternatively, the thiourethane prepolymer composition can be any thiourethane prepolymer composition described as a reaction product described herein. For example the thiourethane prepolymer composition can be described as any reaction product of a catalyst, a thiol ester composition, and an isocyanate composition described herein; or alternatively, as any reaction product of a thiol ester composition, an isocyanate composition, a catalyst, and a property modifying agent composition described herein. Since the catalyst, thiol ester composition, the isocyanate composition, and optionally the property modifying agent (or property modifying agent composition) are independent elements of the thiourethane prepolymer composition utilized to produce the thiourethane polymer, the thiourethane prepolymer composition described as a reaction product of a catalyst, thiol ester composition, isocyanate composition, and optionally the property modifying agent (or property modifying agent composition) can be described using any catalyst described herein, any thiol ester composition described herein, any isocyanate composition described herein, and optionally any property modifying agent (or property modifying agent composition) described herein.

Generally, the thiol ester composition and the isocyanate composition can be combined in any functional group equivalent ratio that can produce a thiourethane prepolymer composition. The functional group equivalent ratio relates the ratio of the number of functional groups in the thiol ester composition capable of reacting with an isocyanate group of the isocyanate composition to form a thiourethane group to the number of isocyanate groups in the isocyanate composition. The functional group equivalent ratio is provided by the term “isocyanate index” or “NCO:XH equivalent ratio” where NCO represents the equivalents of isocyanate groups used to produce the thiourethane prepolymer composition and XH represents the equivalents of active hydrogen groups used to produce the thiourethane prepolymer composition. The active hydrogen groups can include thiol groups, alcohol groups, amine groups, amide groups, carboxylic acid groups, and combinations thereof. In an embodiment, the active hydrogen groups can include alcohol groups, thiol groups, and amine groups; or alternatively, can include alcohol groups and thiols groups; alternatively, comprise thiols groups; alternatively, consisting of alcohol groups and thiol groups; or alternatively, consist essentially of thiol groups. Other suitable active hydrogen groups will be apparent to those of skill in the art and are to be considered within the scope of the present invention. In an embodiment, the XH in the NCO:XH equivalent ratio used to prepare the thiourethane prepolymer consists essentially of thiols group and alcohol groups. In some embodiments, the XH in the NCO:XH equivalent ratio used to prepare the thiourethane prepolymer consist essentially of thiol groups. In an aspect, substantially all of the active hydrogen groups are capped by isocyanate groups. One skilled in the art will further recognize which thiol ester composition comprises only thiol groups and which thiol ester composition comprises thiol groups and alcohol groups. In some embodiments, substantially all of the XH in the NCO:XH equivalent ratio used to prepare the thiourethane prepolymer comes from the thiol ester composition. In other embodiments, a portion of the XH in the NCO:XH equivalent ratio used to prepare the thiourethane prepolymer can come from an added agent (e.g. a property modifying agent such as a polyol or any other compound having an active hydrogen group described herein). When a portion of the active hydrogen groups come from compounds other than the thiol ester, the portion of active hydrogen groups that come from compounds other than the thiol ester are designated as YH and can represent any active hydrogen group, or any combination of active hydrogen groups, described herein. In some embodiments, YH represents an alcohol group. In embodiments, the functional group equivalent ratio (NCO:XH) can be at least 2.1. In some embodiments, the NCO:XH equivalent ratio can range from 2.1 to 10:1; alternatively, from 2.1 to 6.0; or alternatively, from 2.1 to 4.0. In some embodiments, the NCO:XH equivalent ratio can range from 2.5 to 3.5. In some embodiments wherein the thiourethane prepolymer is produced with a property modifying agent, the property modifying agent can be added in an amount to achieve a particular YH:SH molar ratio while maintaining a NCO:XH ratio having any value disclosed herein. In some embodiments, the YH:SH molar ratio can range from 0.01:1 to 5:1; alternatively, range from 0.05:1 to 3:1; alternatively, range from 0.1:1 to 2:1; or alternatively, range from 0.15:1 to 1.6:1.

In an aspect, the method of making the thiourethane polymer composition further comprises curing the thiourethane prepolymer composition using moisture. Heat can also be used to cure the thiourethane prepolymer compositions of the present invention. Other suitable curing profiles will be apparent to those of skill in the art and are to be considered within the scope of the present invention.

In embodiments, the catalyst used to produce the thiourethane prepolymer composition can be selected from the group consisting of a tertiary amine, an organo-tin compound, an amine initiated polypropylene glycol, and combinations thereof. In some embodiments the catalyst can be an amine. In other embodiments, the catalyst can be a tin compound. In some embodiments, the catalyst is a tertiary amine. In other embodiments the catalyst can be aliphatic amine; or alternatively, an aromatic amine. In other embodiments, the catalyst can be a polyether amine; alternatively, a polyalkylene amine; or alternatively, a tertiary amine polyol. In yet other embodiments, the amine catalyst can be a polyamine comprising at least two amine groups. In some amine catalyst embodiment, the catalyst can be 1,8-diazabicyclo[5,4,0]undec-7-ene [DBU-CAS# 6674-22-2]; alternatively, 1,4-diazabicyclo[2.2.2]octane [DABCO-CAS# 280-57-9]); or alternatively, triethylamine. In a tin compound catalyst embodiment, the tin compound can be dibutyl tin dilaurate. Any of these catalysts can be used as the second catalyst to produce the thiourethane polymer composition, such as in the multi-component curing systems.

Generally, the catalyst is utilized when the thiourethane prepolymer composition comprising the thiol ester composition and the isocyanate does not cure under the desired conditions. In embodiments, the catalyst can comprise less than 10 weight percent of the thiourethane prepolymer composition. In other embodiments, the catalyst comprises from 0.01% to 9.0% by weight of the thiourethane prepolymer composition; alternatively, from 0.1% to 7.0% by weight of the thiourethane prepolymer composition; or alternatively, from 0.5% to 3.0% by weight of the thiourethane prepolymer composition.

The processes described herein can be used to produce any of the thiourethane polymer compositions described herein. The order of the process steps can be varied as described herein.

### Feedstocks

### Thiol Ester Composition

The thiol ester composition used as a feedstock to produce the polymers described herein can be described using a number of different methods. Functionally, the thiol ester can be described by the type of functional groups present in the thiol ester. In this functional description, the thiol ester composition minimally contains molecules having at least one ester group and at least one thiol group. In other embodiments, the thiol ester composition can include thiol esters with and without additional groups, such as hydroxy groups, and/or polysulfide linkages —Sₓ— wherein x is an integer greater than 1. When the thiol ester contains the hydroxy group, the thiol ester is referred to as a hydroxy thiol ester. When the thiol ester has polysulfide linkages —Sₓ— wherein x is an integer greater than 1, the thiol ester can be referred to as a crosslinked thiol ester. When the thiol ester has a hydroxy group and a polysulfide group —Sₓ— wherein x is an integer greater than 1, the thiol ester can be referred to as crosslinked hydroxy thiol ester.

Alternatively, the thiol ester can be described using a name that indicates the method by which it was formed. For example, a thiol ester referred to as a mercaptanized unsaturated ester refers to a thiol ester produced by reacting hydrogen sulfide with an unsaturated ester. The mercaptanized unsaturated ester can be further described utilizing the functional descriptors of the thiol esters present in the mercaptanized unsaturated ester. For example, in two non-limiting examples, a mercaptanized soybean oil can be further described by a combination of the number of ester groups and the number of thiol groups among others thiol ester aspects present in the mercaptanized soybean oil, while a mercaptanized castor oil can be further described by a combination of the number of ester groups, the number of thiol groups, the number of hydroxy groups, among other thiol ester aspects present in the mercaptanized castor oil.

In an aspect, the thiol ester composition of the present invention can be produced by reacting any unsaturated ester with hydrogen sulfide, as described in U.S. patent application Ser. Nos. 11/060,675; 11/060,696; 11/059,792; and 11/059,647 (hereinafter “the '675 applications”), the disclosure of which is incorporated by reference in its entirety. When the thiol ester composition is produced by reacting an unsaturated ester with hydrogen sulfide, the material produced can be referred to as the mercaptanized unsaturated ester. Because the unsaturated esters can contain multiple carbon-carbon double bonds per unsaturated ester molecule, carbon-carbon double bond reactivity and statistical probability dictate that each thiol ester molecule of the feedstock thiol ester composition produced from the unsaturated ester composition will not have the same number of thiol groups, number of unreacted carbon-carbon double bonds, number of cyclic sulfides, molar ratio of carbon-carbon double bonds to thiol groups, molar ratio of cyclic sulfides to thiol groups, and/or other quantities of functional groups and molar ratios disclosed herein as the unsaturated ester. Additionally, the unsaturated esters can also comprise a mixture of individual unsaturated esters having a different number of carbon-carbon double bonds and/or ester groups. Thus, many of these properties will be described as an average number of the groups per thiol ester molecule within the thiol ester composition or average ratio per thiol ester molecule within the thiol ester composition. In other embodiments, it is desired to control the thiol sulfur content present in the thiol ester. Because it is difficult to ensure that the hydrogen sulfide reacts with every carbon-carbon double bond within the unsaturated ester, certain molecules of thiol ester can have more or less thiol groups than other molecules. Thus, the weight percent of thiol groups is stated as an average across all thiol ester molecules of the thiol ester composition.

When the thiol ester is cross-linked, the thiol ester is referred to as a cross-linked thiol ester or a cross-linked hydroxy thiol ester, depending upon the compositions used to produce the cross-linked thiol ester. Each of these types of thiol ester compositions are described herein. Hydroxy thiol esters, cross-linked hydroxy thiol esters, mercaptanized unsaturated esters, mercaptanized epoxidized esters, cross-linked mercaptanized unsaturated esters, and cross-linked mercaptanized epoxidized esters are all considered to be thiol ester compositions. Many of the same attributes that are used to describe the thiol ester compositions, such as the molar ratios and other independent descriptive elements described herein, are equally applicable to many of the different types of thiol ester compositions described herein.

Generally, the thiol ester compositions can be described as comprising one or more separate or discreet functional groups of the thiol ester molecule and/or thiol ester composition. These independent functional groups can include: the number of (or average number of) ester groups per thiol ester molecule, the number of (or average number of) thiol groups per thiol ester molecule, the number of (or average number of) unreacted carbon-carbon double bonds per thiol ester molecule, the average thiol sulfur content of the thiol ester composition, the percentage (or average percentage) of sulfide linkages per thiol ester molecule, and the percentage (or average percentage) of cyclic sulfide groups per thiol ester molecule. Additionally, the thiol ester compositions can be described using individual or a combination of ratios including the ratio of double bonds to thiol groups, the ratio of cyclic sulfides to mercaptan groups, and the like. As separate elements, these functional groups of the thiol composition will be described separately.

Minimally, the thiol ester contains thiol ester molecules having at least one ester group and one thiol group per thiol ester molecule. In embodiments, the thiol ester can be prepared from unsaturated esters. Therefore, in some embodiments, the thiol ester can contain the same number of ester groups as the unsaturated esters from which they are prepared, as described herein. In an embodiment, the thiol ester molecules can have an average of at least 1.5 ester groups per thiol ester molecule. Alternatively, the thiol ester molecules can have an average of at least 2 ester groups per thiol ester molecule; alternatively, an average of at least 2.5 ester groups per thiol ester molecule; or alternatively, an average of at least 3 ester groups per thiol ester molecule. In other embodiments, the thiol esters can have an average of from 1.5 to 8 ester groups per thiol ester molecule; alternatively, an average of from 2 to 7 ester groups per thiol ester molecule; alternatively, an average of from 2.5 to 5 ester groups per thiol ester molecule; or alternatively, an average of from 3 to 4 ester groups per thiol ester molecule. In yet other embodiments, the thiol ester can comprise an average of about 3 ester groups per thiol ester molecule or alternatively, an average of about 4 ester groups per thiol ester molecule.

Minimally, the thiol ester comprises one or an average of at least one thiol group per thiol ester molecule. In an embodiment, the thiol ester molecules can have an average of at least 1.5 thiol groups per thiol ester molecule; alternatively, an average of at least 2 thiol groups per thiol ester molecule; alternatively, an average of at least 2.5 thiol groups per thiol ester molecule; or alternatively, an average of at least 3 thiol groups per thiol ester molecule. In other embodiments, the thiol ester molecules can have an average of from 1.5 to 9 thiol groups per thiol ester molecule; alternatively, an average of from 3 to 8 thiol groups per thiol ester molecule; alternatively, an average of from 2 to 4 thiol groups per thiol ester molecule; or alternatively, an average of from 4 to 8 thiol groups per thiol ester molecule.

In an aspect, the thiol ester can be described using the number of thiol groups present in the thiol ester. For example, a thiol ester referred to as a trimercaptan thiol ester can be a thiol ester containing an average of between 2.5 to 3.5 thiol groups per thiol ester molecule. Alternatively, the trimercaptan thiol ester can contain an average of between 2.75 to 3.25 thiol groups per thiol ester molecule. As another example, a thiol ester referred to as a dimercaptan thiol ester can be a thiol ester containing an average of between 1.5 to 2.5 thiol groups per thiol ester molecule; or alternatively, between 1.75 and 2.25 thiol groups per thiol ester molecule.

In other embodiments, the thiol ester can be further described by the average amount of thiol sulfur present in the thiol ester. In an embodiment, the thiol ester molecules have an average of at least 5 weight percent thiol sulfur per thiol ester molecule; alternatively, an average of at least 10 weight percent thiol sulfur per thiol ester molecule; or alternatively, an average of greater than 15 weight percent thiol sulfur per thiol ester molecule. In an embodiment, the thiol ester molecules have an average of from 5 to 25 weight percent thiol sulfur per thiol ester molecule; alternatively, an average of from 5 to 20 weight percent thiol sulfur per thiol ester molecule; alternatively, an average of from 6 to 15 weight percent thiol sulfur per thiol ester molecule; or alternatively, an average of from 8 to 10 weight percent thiol sulfur per thiol ester molecule.

Generally, the location of the thiol group of the thiol ester is not particularly important and will be dictated by the method used to produce the thiol ester. In embodiments wherein the thiol ester is produced by contacting an unsaturated ester with hydrogen sulfide (a mercaptanized unsaturated ester), the position of the thiol group will be dictated by the position of the carbon-carbon double bond. When the carbon-carbon double bond is an internal carbon-carbon double bond, the method of producing the thiol ester will result in a secondary thiol group. However, when the double bond is located at a terminal position it is possible to choose reaction conditions to produce a thiol ester comprising either a primary thiol group or a secondary thiol group.

Some methods of producing the thiol ester composition can additionally create sulfur containing functional groups other than a thiol group. For example, in some thiol ester production methods, an introduced thiol group can react with a carbon-carbon double bond within the same unsaturated ester to produce a sulfide linkage. When the reaction is with a double bond of a second unsaturated ester, a simple sulfide linkage is produced. However, in some instances, the second carbon-carbon double bond is located in the same unsaturated ester molecule. When the thiol group reacts with a second carbon-carbon double bond within the same unsaturated ester molecule, a sulfide linkage is produced. In some instances, the carbon-carbon double bond can be within a second ester group of the unsaturated ester molecule. While in other instances, the carbon-carbon double bond can be within the same ester group of the unsaturated ester molecule.

When the thiol group reacts with the carbon-carbon double bond in a second ester group of the same unsaturated ester molecule, the sulfide contains at least one ester group within a ring structure. In some embodiments when the thiol group reacts with the carbon-carbon double bond in a second ester group of the same unsaturated ester molecule, the sulfide contains two ester groups within a ring structure. Within this specification, the first type of sulfide containing an ester group within the ring structure is referred to as a simple sulfide. When the thiol group reacts with the carbon-carbon double bond within the same ester group, the sulfide does not contain an ester group within the ring structure. Within this specification, this second type of sulfide is referred to as a cyclic sulfide. In the cyclic sulfide case, the sulfide linkage produces a cyclic sulfide functionality within a single ester group of the thiol ester. The cyclic sulfide rings that can be produced include a tetrahydrothiopyran ring, a thietane ring, or a thiophane ring (tetrahydrothiophene ring).

In some embodiments, it is desirable to control the average amount of sulfur present as cyclic sulfide in the thiol ester. In an embodiment, the average amount of sulfur present as cyclic sulfide in the thiol ester molecules comprises less than 30 mole percent. Alternatively, the average amount of sulfur present as cyclic sulfide in the thiol esters can comprise less than 20 mole percent; alternatively, less than 10 mole percent; alternatively, less than 5 mole percent; or alternatively, less than 2 mole percent. In other embodiments, it is desirable to control the molar ratio of cyclic sulfides to thiol groups. In an embodiment, the average molar ratio of cyclic sulfide groups to thiol groups per thiol ester can be less than 1.5. Alternatively, the average molar ratio of cyclic sulfide groups to thiol groups per thiol ester can be less than 1; alternatively, less than 0.5; alternatively, less than 0.25; or alternatively, less than 0.1. In some embodiments, the ratio of cyclic sulfide groups to thiol groups per thiol ester can range from 0 to 1; or alternatively, the average molar ratio of cyclic sulfide groups to thiol groups per thiol ester can range between 0.05 and 1.

In some instances, it is desirable to have carbon-carbon double bonds present in the thiol ester composition, while in other embodiments, it can be desirable to minimize the number of carbon-carbon double bonds present in the thiol ester composition. The presence of carbon-carbon double bonds in the thiol ester can be stated as an average molar ratio of carbon-carbon double bonds to thiol-sulfur. In an embodiment, the average ratio of the remaining unreacted carbon-carbon double bond in the thiol ester composition to thiol sulfur can be less than 1.5 per thiol ester molecule. Alternatively, the average ratio of carbon-carbon double bond to thiol sulfur can be less than 1.2 per thiol ester molecule; alternatively, less than 1.0 per thiol ester molecule; alternatively, less than 0.75 per thiol ester molecule; alternatively, less than 0.5 per thiol ester molecule; alternatively, less than 0.2 per thiol ester molecule; or alternatively, less than 0.1 per thiol ester molecule.

In particular embodiments, the thiol ester is produced from unsaturated ester compositions (a mercaptanized unsaturated ester). Because the unsaturated ester has particular compositions having a certain number of ester groups present, the product thiol ester composition will have about the same number of ester groups per thiol ester molecule as the unsaturated ester. Other, independent thiol ester properties are described herein can be used to further describe the thiol ester composition.

In an aspect, the thiol ester can be referred to as a mercaptanized unsaturated ester. In these embodiments, the unsaturated ester described herein and/or the unsaturated ester functional descriptions described herein can be utilized to further indicate and/or further describe a particular mercaptanized ester. In a few non-limiting examples, the thiol ester produced by contacting a natural source oil with hydrogen sulfide can be referred to as mercaptanized natural source oil, the thiol ester produced by contacting a soybean oil with hydrogen sulfide can be referred to as mercaptanized soybean oil, and the thiol ester produced by contacting a castor oil with hydrogen sulfide can be referred to as mercaptanized castor oil. Additional properties of the unsaturated ester oils described herein can also be utilized to further describe the unsaturated ester oil and the mercaptanized ester oil.

In some embodiments, the thiol ester molecules are produced from unsaturated esters having an average of less than 25 weight percent of side chains having 3 contiguous methylene interrupted carbon-carbon double bonds, as described herein. In some embodiments, greater than 40 percent of the thiol ester molecule total side chains can include sulfur. In some embodiments, greater than 60 percent of the thiol ester molecule total side chains can include sulfur. In other embodiments, greater than 50, 70, or 80 percent of the thiol ester molecule total side chains can include sulfur.

In an embodiment, the thiol ester is a thiol containing natural source oil, as described herein. When the thiol ester is a thiol containing natural source oil, functional groups that are present in the thiol containing natural source oil can be described in a “per thiol ester molecule” basis or in a “per triglyceride” basis. The thiol containing natural source oil can have substantially the same properties as the thiol ester composition, such as the molar ratios and other independent descriptive elements described herein.

The average number of thiol groups per triglyceride in the thiol containing natural source oil is greater than about 1.5. In some embodiments, the average number of thiol groups per triglyceride can range from about 1.5 to about 9.

The mercaptanized unsaturated ester composition can also be described as a product produced by the process comprising contacting hydrogen sulfide and an unsaturated ester composition. In other words, the unsaturated ester composition is mercaptanized to form the mercaptanized unsaturated ester composition. The mercaptanized unsaturated ester composition can also be described using a molecular weight or an average molecular weight of the side chains. All of the attributes used to describe the thiol ester composition can be used to describe the mercaptanized unsaturated ester composition.

### Hydroxy Thiol Ester Composition

In an aspect, the thiol ester composition used as a feedstock to produce the polymers described herein can be a hydroxy thiol ester. The hydroxy thiol ester can be described using a number of methods. Functionally, the hydroxy thiol ester can be described by the types of functional groups present in the hydroxy thiol ester. In this functional description, the hydroxy thiol ester composition minimally contains molecules having at least one ester group, at least one thiol group, and at least one hydroxy group. In other embodiments, the thiol ester composition can include thiol esters with and without additional groups, polysulfide linkages —Sₓ— wherein x is an integer greater than 1. When the thiol ester has a hydroxy group and a polysulfide group —Sₓ— wherein x is an integer greater than 1, the thiol ester can be referred to as crosslinked hydroxy thiol ester.

Alternatively, the hydroxy thiol ester can be described using a name that indicates the method by which it was formed. For example, a hydroxy thiol ester that is called a mercaptanized epoxidized ester refers to a hydroxy thiol ester produced by reacting hydrogen sulfide with an epoxidized unsaturated ester. The mercaptanized epoxidized ester can be further described utilizing the function descriptor of the hydroxy thiol ester present in the mercaptanized epoxidized ester. Compounds that fit the hydroxy thiol ester composition description do not always fit the mercaptanized epoxidized ester description. For example, while mercaptanized castor oil can be described using some of the hydroxy thiol ester definitions by virtue of having at least one ester group, at least one thiol group, and at least one hydroxy group. Mercaptanized castor oil, however, is not a mercaptanized epoxidized ester as it is produced by contacting castor oil with hydrogen sulfide. However, mercaptanized epoxidized castor oil is a mercaptanized epoxidized ester oil by virtue of its formation by contacting hydrogen sulfide with epoxidized castor oil.

The feedstock thiol ester compositions can also contain a hydroxy or alcohol group. When the thiol ester composition includes the hydroxy group, the thiol ester composition is referred to herein as the hydroxy thiol ester composition. The quantity or number of alcohol groups present in the hydroxy thiol ester composition can be independent of the quantity of other functional groups present in the hydroxy thiol ester composition (i.e. thiol groups, ester groups, sulfides, cyclic sulfides). Additionally, the weight percent of thiol sulfur and functional group ratios (i.e. molar ratio of cyclic sulfides to thiol groups, molar ratio of epoxide groups to thiol groups, molar ratio of epoxide groups to α-hydroxy thiol groups and other disclosed quantities of functional groups and their molar ratios to the thiol groups) are separate or discreet elements that can be used to describe the hydroxy thiol ester composition. The hydroxy thiol ester composition can be described using any combination of the hydroxy thiol ester composition separate functional groups or ratios described herein.

In an embodiment, the hydroxy thiol ester composition can be produced by reacting hydrogen sulfide with an epoxidized unsaturated ester composition as described in the '675 applications. When the thiol ester composition is produced by reacting hydrogen sulfide with an epoxidized unsaturated ester, the material produced can be called a mercaptanized epoxidized ester. Because the epoxidized unsaturated ester can contain multiple epoxide groups, epoxide group reactivity and statistical probability dictate that not all hydroxy thiol ester molecules of the hydroxy thiol ester composition will have the same number of hydroxy groups, thiol groups, α-hydroxy thiol groups, sulfides, cyclic sulfides, molar ratio of cyclic sulfides to thiol groups, molar ratio of epoxide groups to thiol groups, molar ratio of epoxide groups to α-hydroxy thiol groups, weight percent thiol sulfur, and/or other disclosed quantities of functional groups and their molar ratios as the epoxidized unsaturated ester composition. Thus, many of these properties will be discussed as an average number or ratio per hydroxy thiol ester molecule. In other embodiments, it is desired to control the content of thiol sulfur present in the hydroxy thiol ester. Because it is difficult to ensure that the hydrogen sulfide reacts with every epoxide group within the epoxidized unsaturated ester, certain hydroxy thiol ester molecules can have more or less thiol groups than other molecules within the hydroxy thiol ester composition. Thus, the weight percent of thiol groups can be stated as an average weight percent across all hydroxy thiol ester molecules.

As an embodiment of the present invention, the hydroxy thiol ester composition includes hydroxy thiol ester molecules that have an average of at least 1 ester group and an average of at least 1α-hydroxy thiol group per hydroxy thiol ester molecule. As an embodiment of the present invention, the hydroxy thiol ester composition includes hydroxy thiol ester molecules that have an average of at least 1.5 ester groups and an average of at least 1.5α-hydroxy thiol groups per hydroxy thiol ester molecule.

Alternatively, in some embodiments, the hydroxy thiol ester comprises at least one ester, at least one thiol group, and at least one hydroxy group. Thus, in some embodiments, the hydroxy thiol ester composition includes hydroxy thiol ester molecules that have an average of at least 1.5 ester groups, an average of at least one thiol group, and an average of at least 1.5 hydroxy groups per hydroxy thiol molecule.

In embodiments the hydroxy thiol ester can be prepared from the epoxidized unsaturated ester or the unsaturated ester. Thus, the hydroxy thiol ester can contain the same number of ester groups as the epoxidized unsaturated ester or unsaturated ester. In an embodiment, the hydroxy thiol ester molecules have an average of at least 1.5 ester groups per hydroxy thiol ester molecule. Alternatively, the hydroxy thiol ester molecules have an average of at least 2 ester groups per hydroxy thiol ester molecule; alternatively, an average of at least 2.5 ester groups per hydroxy thiol ester molecule; or alternatively, an average of at least 3 ester groups per hydroxy thiol ester molecule. In other embodiments, the hydroxy thiol esters have an average of from 1.5 to 8 ester groups per hydroxy thiol ester molecule; alternatively, an average of from 2 to 7 ester groups per hydroxy thiol ester molecule; alternatively, an average of from 2.5 to 5 ester groups per hydroxy thiol ester molecule; or alternatively, an average of from 3 to 4 ester groups per hydroxy thiol ester molecule. In yet other embodiments, the hydroxy thiol ester comprises an average of 3 ester groups per hydroxy thiol ester molecule; or alternatively, an average of 4 ester groups per hydroxy thiol ester molecule.

In some embodiments, the hydroxy group and the thiol group are combined in the same group (e.g. when the hydroxy thiol ester is produced from the epoxidized unsaturated ester), the group can be referred to as an α-hydroxy thiol group. In other embodiments, the thiol group and the hydroxy or alcohol group are not in the same group. In this instance, the presence of the alcohol group is not dependent upon the formation of the thiol group. For example, as another embodiment of the present invention, the hydroxy thiol ester composition includes hydroxy thiol ester molecules. The hydroxy thiol ester molecules have an average of at least 1.5 ester groups, an average of at least 1.5 thiol groups, and an average of at least 1.5 alcohol groups per hydroxy thiol ester molecule.

Minimally, in some embodiments, the hydroxy thiol ester comprises at least one thiol group per hydroxy thiol ester molecule. In an embodiment, the hydroxy thiol ester molecules can have an average of at least 1.5 thiol groups per hydroxy thiol ester molecule; alternatively, an average of at least 2 thiol groups per hydroxy thiol ester molecule; alternatively, an average of at least 2.5 thiol groups per hydroxy thiol ester molecule; or alternatively, an average of at least 3 thiol groups per hydroxy thiol ester molecule. In other embodiments, the hydroxy thiol ester molecules can have an average of from 1.5 to 9 thiol groups per hydroxy thiol ester molecule; alternatively, an average of from 3 to 8 thiol groups per hydroxy thiol ester molecule; alternatively, an average of from 2 to 4 thiol groups per hydroxy thiol ester molecule; or alternatively, an average of from 4 to 8 thiol groups per hydroxy thiol ester.

Minimally, in some embodiments, the hydroxy thiol ester composition comprises an average of at least 1 hydroxy or alcohol group per hydroxy thiol ester molecule. In some embodiments, the hydroxy thiol ester composition can have an average of at least 1.5 hydroxy groups per hydroxy thiol ester molecule; alternatively, an average of at least 2 hydroxy groups per hydroxy thiol ester molecule; alternatively, an average of at least 2.5 hydroxy groups per hydroxy thiol ester molecule; or alternatively, an average of at least 3 hydroxy groups per hydroxy thiol ester molecule. In other embodiments, the thiol ester composition can have an average of from 1.5 to 9 hydroxy groups per hydroxy thiol ester molecule; alternatively, an average of from 3 to 8 hydroxy groups per hydroxy thiol ester molecule; alternatively, an average of from 2 to 4 hydroxy groups per hydroxy thiol ester molecule; or alternatively, an average of from 4 to 8 hydroxy groups per hydroxy thiol ester molecule.

In yet other embodiments, the number of hydroxy groups can be stated as an average molar ratio of hydroxy groups to thiol groups. Minimally, in some embodiments, the molar ratio of hydroxy groups to thiol groups can be at least 0.25. In some embodiments, the molar ratio of hydroxy groups to thiol groups can be at least 0.5; alternatively, at least 0.75; alternatively, at least 1.0; alternatively, at least 1.25; or alternatively, at least 1.5. In other embodiments, the molar ratio of hydroxy groups to thiol groups can range from 0.25 to 2.0; alternatively, from 0.5 to 1.5; or alternatively, from 0.75 to 1.25.

In embodiments where the hydroxy thiol esters are produced from an epoxidized unsaturated ester, the hydroxy thiol esters can be described as containing ester groups and α-hydroxy thiol groups. In this case, the hydroxy thiol esters that contain ester groups and α-hydroxy thiol groups can be referred to as mercaptanized epoxidized esters. The number of ester groups and the number of α-hydroxy thiol groups are independent elements and, as such, the hydroxy thiol esters can be described as having any combination of ester groups and α-hydroxy thiol groups described herein. Minimally, the hydroxy thiol ester can have an average of at least 1α-hydroxy thiol group per hydroxy thiol ester molecule. In some embodiments, the hydroxy thiol ester composition can have an average of at least 1.5α-hydroxy thiol groups per hydroxy thiol ester molecule; alternatively, an average of at least 2α-hydroxy thiol groups per hydroxy thiol ester molecule; alternatively, an average of at least 2.5α-hydroxy thiol groups per hydroxy thiol ester molecule; or alternatively, an average of at least 3α-hydroxy thiol groups per hydroxy thiol ester molecule. In other embodiments, the hydroxy thiol ester composition can have an average of from 1.5 to 9α-hydroxy thiol groups per hydroxy thiol ester molecule; alternatively, an average of from 3 to 8 α-hydroxy thiol groups per hydroxy thiol ester molecule; alternatively, an average of from 2 to 4α-hydroxy thiol groups per hydroxy thiol ester molecule; or alternatively, an average of from 4 to 8α-hydroxy thiol groups per hydroxy thiol ester molecule.

In an aspect, the hydroxy thiol ester can be described using the number of thiol groups or α-hydroxy thiol groups present in the hydroxy thiol ester. For example, a hydroxy thiol ester referred to as a trimercaptan hydroxy thiol ester can be a hydroxy thiol ester containing an average of between 2.5 to 3.5 thiol or α-hydroxy thiol groups per hydroxy thiol ester molecule. Alternatively, the trimercaptan hydroxy thiol ester can contain an average of between 2.75 to 3.25 thiol or α-hydroxy thiol groups per hydroxy thiol ester molecule. As another example, a hydroxy thiol ester referred to as a dimercaptan hydroxy thiol ester can be a hydroxy thiol ester containing an average of between 1.5 to 2.5 thiol or α-hydroxy thiol groups per hydroxy thiol ester molecule; or alternatively, between 1.75 and 2.25 thiol or α-hydroxy thiol groups per hydroxy thiol ester molecule.

In another aspect, the hydroxy thiol ester can be described using the number of alcohol, α-hydroxy thiol, or other functional groups present in the hydroxy thiol ester. For example, a hydroxy thiol ester referred to as a trifunctional hydroxy thiol ester can be a hydroxy thiol ester containing an average of between 2.5 to 3.5 alcohol, α-hydroxy thiol, or other functional groups per hydroxy thiol ester molecule. Alternatively, the trifunctional hydroxy thiol ester can contain an average of between 2.75 to 3.25 alcohol, α-hydroxy thiol, or other functional groups per hydroxy thiol ester molecule. As another example, a hydroxy thiol ester referred to as a difunctional hydroxy thiol ester can be a hydroxy thiol ester containing an average of between 1.5 to 2.5 alcohol, α-hydroxy thiol, or other functional groups per hydroxy thiol ester molecule; or alternatively, between 1.75 and 2.25 alcohol, α-hydroxy thiol, or other functional groups per hydroxy thiol ester molecule.

The hydroxy thiol esters can be produced by contacting an epoxidized ester derived from an unsaturated ester (i.e., epoxidized unsaturated ester) with hydrogen sulfide, as described herein. In some instances, it is desirable to have epoxide groups present in the hydroxy thiol ester composition. While in other embodiments, it can be desirable to minimize the number of epoxy groups present in the hydroxy thiol ester composition. Thus, the presence of residual epoxide groups can be another separate functional group used to describe the hydroxy thiol ester. The hydroxy thiol esters can include an average of greater than 0 to about 4 epoxide groups per triglyceride. The thiol composition can also include an average of greater than 1.5 to about 9 epoxide groups per triglyceride.

The presence of epoxide groups in the hydroxy thiol ester can be independently described as an average number of epoxide groups per hydroxy thiol ester, a molar ratio of epoxide groups to thiol groups, a molar ratio of epoxide groups to α-hydroxy thiol groups, or any combination thereof. In some embodiments, the hydroxy thiol ester molecules can have an average of less than 2 epoxide groups per hydroxy thiol ester molecule, i.e., the hydroxy thiol ester molecules have a molar ratio of epoxide groups to α-hydroxy thiol groups of less than 2. Alternatively, the hydroxy thiol ester can have an average of less than 1.5 epoxide groups per hydroxy thiol ester molecule; alternatively, an average of less than 1 epoxide group per hydroxy thiol ester molecule; alternatively, an average of less than 0.75 epoxide groups per hydroxy thiol ester molecule; or alternatively, an average of less than 0.5 epoxide groups per hydroxy thiol ester molecule. In other embodiments, the molar ratio of epoxide groups to thiol groups can average less than 1.5. Alternatively, the molar ratio of epoxide groups to thiol groups can average less than 1; alternatively, average less than 0.75; alternatively, average less than 0.5; alternatively, average less than 0.25; or alternatively, average less than 0.1. In yet other embodiments, the molar ratio of epoxide groups to α-hydroxy thiol groups can average less than 1.5. Alternatively, the molar ratio of epoxide groups to α-hydroxy thiol groups can average less than 1; alternatively, can average less than 0.75; alternatively, average less than 0.5; alternatively, average less than 0.25; or alternatively, average less than 0.1. In yet other embodiments, the hydroxy thiol ester composition is substantially free of epoxide groups.

In other embodiments, the hydroxy thiol ester can be described by the average amount of thiol sulfur present in the hydroxy thiol ester. In an embodiment, the hydroxy thiol ester molecules can have an average of at least 2.5 weight percent thiol sulfur per hydroxy thiol ester molecule; alternatively, an average of at least 5 weight percent thiol sulfur per hydroxy thiol ester molecule; alternatively, an average of at least 10 weight percent thiol sulfur per hydroxy thiol ester molecule; or alternatively, an average of greater than 15 weight percent thiol sulfur per hydroxy thiol ester molecule. In an embodiment, the hydroxy thiol ester molecules can have an average of from 5 to 25 weight percent thiol sulfur per hydroxy thiol ester molecule; alternatively, an average of from 5 to 20 weight percent thiol sulfur per hydroxy thiol ester molecule; alternatively, an average of from 6 to 15 weight percent thiol sulfur per hydroxy thiol ester molecule; or alternatively, an average of from 8 to 10 weight percent thiol sulfur per hydroxy thiol ester molecule.

In some embodiments, at least 20 percent of the total side chains include the α-hydroxy thiol group. In some embodiments, at least 40 percent of the total side chains include the α-hydroxy thiol group. In some embodiments, at least 60 percent of the total side chains include the α-hydroxy thiol group; or alternatively, at least 70 percent of the total side chains include the α-hydroxy thiol group. Yet in other embodiments, at least 80 percent of the total side chains include the α-hydroxy thiol group.

In some aspects, greater than 20 percent of the hydroxy thiol ester molecule total side chains contain sulfur. In some aspects, greater than 40 percent of the hydroxy thiol ester molecule total side chains contain sulfur. In some aspects, greater than 60 percent of the hydroxy thiol ester molecule total side chains contain sulfur; alternatively, greater than 70 percent of the total side chains contain sulfur; or alternatively, greater than 80 percent of the total side chains contain sulfur.

In particular embodiments, the epoxidized unsaturated ester used in the synthesis of the hydroxy thiol ester is produced from the epoxidized unsaturated ester composition that includes an epoxidized natural source oil. Because the natural source oils have particular compositions regarding the number of ester groups present, the hydroxy thiol ester will have about the same number of ester groups as the natural source oil. Other independent properties that are described herein can be used to further describe the hydroxy thiol ester.

In other embodiments, the epoxidized unsaturated ester used to produce the hydroxy thiol ester is produced from synthetic (or semi-synthetic) unsaturated ester oils. Because the synthetic ester oils can have particular compositions regarding the number of ester groups present, the hydroxy thiol ester would have about the same number of ester groups as the synthetic ester oil. Other independent properties of the unsaturated ester, whether the unsaturated ester includes natural source or synthetic oils, can be used to further describe the hydroxy thiol ester composition.

Examples of suitable hydroxy thiol esters include but are not limited to mercaptanized epoxidized vegetable oils, mercaptanized epoxidized soybean oil, and mercaptanized epoxidized castor oil. Other suitable mercaptanized epoxidized esters are described in the '675 applications and are to be considered within the scope of the present invention.

### Cross-Linked Thiol Ester Compositions

In an aspect, the feedstock thiol ester compositions include a cross-linked thiol ester composition. Generally, the cross-linked thiol ester molecules are oligomers of thiol esters that are connected together by polysulfide linkages —Sₓ— wherein x is an integer greater than 1. As the cross-linked thiol ester is described as an oligomer of thiol esters, the thiol esters can be described as the monomer from which the cross-linked thiol esters are produced. In embodiments, the cross-linked thiol ester is produced from a mercaptanized unsaturated ester and can be called a cross-linked mercaptanized unsaturated ester. In other embodiments, the cross-linked thiol ester can be produced from a hydroxy thiol ester and can be called a crossed linked hydroxy thiol ester. In yet other embodiments, the crosslinked thiol ester can be produced from a mercaptanized epoxidized ester and can be called a cross-linked mercaptanized epoxidized thiol ester.

In an aspect, the cross-linked thiol ester composition comprises a thiol ester oligomer having at least two thiol ester monomers connected by a polysulfide linkage having a structure —S_{Q}—, wherein Q is an integer greater than 1. In an aspect, the polysulfide linkage can be the polysulfide linkage —S_{Q}—, wherein Q is 2, 3, 4, or mixtures thereof. In other embodiments, Q can be 2; alternatively, 3; or alternatively, 4.

In an aspect, the cross-linked thiol ester composition comprises a thiol ester oligomer having at least 3 thiol ester monomers connected by polysulfide linkages; alternatively, at least 5 thiol ester monomers connected by polysulfide linkages; alternatively, at least 7 thiol ester monomers connected by polysulfide linkages; or alternatively, at least 10 thiol ester monomers connected by polysulfide linkages. In yet other embodiments, the cross-linked thiol ester composition comprises a thiol ester oligomer having from 3 to 20 thiol ester monomers connected by polysulfide linkages; alternatively, from 5 to 15 thiol ester monomers connected by polysulfide linkages; or alternatively, from 7 to 12 thiol ester monomers connected by polysulfide linkages.

In an aspect, the cross-linked thiol ester composition comprises thiol ester monomers and thiol ester oligomers. In some embodiments, the cross-linked thiol ester composition has a combined thiol ester monomer and thiol ester oligomer average molecular weight greater than 2,000. In other embodiments, the cross-linked thiol ester composition has a combined thiol ester monomer and thiol ester oligomer average molecular weight greater than 5,000; or alternatively, greater than 10,000. In yet other embodiments, the cross-linked thiol ester composition has a combined thiol ester monomer and thiol ester oligomer average molecular weight ranging from 2,000 to 20,000; alternatively, from 3,000 to 15,000; or alternatively, from 7,500 to 12,500.

In an aspect, the thiol ester monomers and thiol ester oligomers have a total thiol sulfur content greater than 0.5 weight percent. In other embodiments, the thiol ester monomers and thiol ester oligomers have a total thiol sulfur content greater than 1 weight percent; alternatively, greater than 2 weight percent; or alternatively, greater than 4 weight percent. In yet other embodiments, the thiol ester monomers and the thiol ester oligomers have a total thiol sulfur content from 0.5 weight percent to 8 weight percent; alternatively, from 4 weight percent to 8 weight percent; or alternatively, 0.5 weight percent to 4 weight percent.

In an aspect, the thiol ester monomers and thiol ester oligomers have a total sulfur content greater than 8 weight percent. In some embodiments, the thiol ester monomers and thiol ester oligomers have a total sulfur content greater than 10 weight percent; or alternatively, greater than 12 weight percent. In yet other embodiments, the thiol ester monomers and thiol ester oligomers have a total sulfur content ranging from 8 weight percent to 15 weight percent; alternatively, from 9 weight percent to 14 weight percent; or alternatively, from 10 weight percent to 13 weight percent.

In an aspect the crosslinked thiol esters of the crosslinked thiol ester compositions can be described as being high crosslinked, mid crosslinked, or low crosslinked. Generally, the amount of crosslinking in the crosslinked thiol esters can be controlled by the amount of sulfur utilized in the production of the crosslinked thiol esters; i.e. the higher the quantity of sulfur utilized in the production of the crosslinked thiol ester, the greater the crosslinking in the crosslinked thiol ester composition. Because the elemental sulfur reacts with the thiol group of the thiol ester composition, the amount of crosslinking can be determined by measuring the residual thiol sulfur content of the remaining in the thiol ester composition.

In embodiments, a low crosslinked thiol ester composition can have an average of from 4.5 to 7.5 weight percent thiol sulfur; alternatively, from 5.0 to 7.0 weight percent thiol sulfur; or alternatively, from 5.5 to 6.5 weight percent thiol sulfur. In embodiments, a mid crosslinked thiol ester composition can have an average of from 2.5 to 3.5 weight percent thiol sulfur; or alternatively, from 2.25 to 2.75 weight percent thiol sulfur. In embodiments, a high crosslinked thiol ester composition can have an average of from 0.75 to 2.25 weight percent thiol sulfur; alternatively, from 1.0 to 2.0 weight percent thiol sulfur; or alternatively, from 1.25 to 1.75 weight percent thiol sulfur.

### Unsaturated Esters

The unsaturated ester used as a feedstock to produce some of the thiol ester compositions described herein can be described using a number of different methods. One method of describing the unsaturated ester feedstock is by the number of ester groups and the number of carbon-carbon double bonds that comprise each unsaturated ester oil molecule. Suitable unsaturated esters used as a feedstock to produce the thiol ester compositions described herein minimally comprise at least 1 ester group and at least 1 carbon-carbon double bond. However, beyond this requirement, the number of ester groups and carbon-carbon double bonds comprising the unsaturated esters are independent elements and can be varied independently of each other. Thus, the unsaturated esters can have any combination of the number of ester groups and the number of carbon-carbon double bonds described separately herein. Suitable, unsaturated esters can also contain additional functional groups such as alcohol, aldehyde, ketone, epoxy, ether, aromatic groups, and combinations thereof. As an example, the unsaturated esters can also comprise hydroxy groups. An example of an unsaturated ester that contains hydroxy groups is castor oil. Other suitable unsaturated esters will be apparent to those of skill in the art and are to be considered within the scope of the present invention.

Minimally, the unsaturated ester comprises at least one ester group. In other embodiments, the unsaturated ester comprises at least 2 ester groups. Alternatively, the unsaturated ester comprises 3 ester groups. Alternatively, the unsaturated ester comprises 4 ester groups. Alternatively, the unsaturated ester includes from 2 to 8 ester groups. Alternatively, the unsaturated ester includes from 2 to 7 ester groups. Alternatively, the unsaturated ester includes from 3 to 5 ester groups. As another alternative, the unsaturated ester includes from 3 to 4 ester groups.

In other embodiments, the unsaturated ester comprises a mixture of unsaturated esters. In these situations, the number of ester groups is best described as an average number of ester groups per unsaturated ester molecule comprising the unsaturated ester composition. In some embodiments, the unsaturated esters have an average of at least 1.5 ester groups per unsaturated ester molecule; alternatively, an average of at least 2 ester groups per unsaturated ester molecule; alternatively, an average of at least 2.5 ester groups per unsaturated ester molecule; or alternatively, an average of at least 3 ester groups per unsaturated ester molecule. In other embodiments, the unsaturated esters have an average of from 1.5 to 8 ester groups per unsaturated ester molecule; alternatively, an average of from 2 to 7 ester groups per unsaturated ester molecule; alternatively, an average of from 2.5 to 5 ester groups per unsaturated ester molecule; or alternatively, an average of from 3 to 4 ester groups per unsaturated ester molecule. In another embodiment, the unsaturated esters have an average of about 3 ester groups per unsaturated ester molecule; or alternatively, an average of about 4 ester groups per unsaturated ester molecule.

Minimally, the unsaturated ester comprises at least one carbon-carbon double bond per unsaturated ester molecule. In an embodiment, the unsaturated ester comprises at least 2 carbon-carbon double bonds; alternatively, at least 3 carbon-carbon double bonds; or alternatively, at least 4 carbon-carbon double bonds. In other embodiments, the unsaturated ester comprises from 2 to 9 carbon-carbon double bonds; alternatively, from 2 to 4 carbon-carbon double bonds; alternatively, from 3 to 8 carbon-carbon double bonds; or alternatively, from 4 to 8 carbon-carbon double bonds.

In some embodiments, the unsaturated ester comprises a mixture of unsaturated esters. In this aspect, the number of carbon-carbon double bonds in the mixture of unsaturated ester is best described as an average number of carbon-carbon double bonds per unsaturated ester oil molecule. In some embodiments, the unsaturated esters have an average of at least 1.5 carbon-carbon double bonds per unsaturated ester molecule; alternatively, an average of at least 2 carbon-carbon double bonds per unsaturated ester molecule; alternatively, an average of at least 2.5 carbon-carbon double bonds per unsaturated ester molecule; or alternatively, an average of at least 3 carbon-carbon double bonds per unsaturated ester molecule. In other embodiments, the unsaturated esters have average of from 1.5 to 9 carbon-carbon double bonds per unsaturated ester molecule; alternatively, an average of from 3 to 8 carbon-carbon double bonds per unsaturated ester molecule; alternatively, an average of from 2 to 4 carbon-carbon double bonds per unsaturated ester molecule; or alternatively, an average of from 4 to 8 carbon-carbon double bonds per unsaturated ester molecule.

While the number (or average number) of ester groups and the number (or average number) double bonds are independent elements of the unsaturated esters, particular embodiments are mentioned for illustrative purposes. In an embodiment, the unsaturated ester molecules have an average of at least 1.5 ester groups per unsaturated ester molecule and have an average of at least 1.5 carbon-carbon double bonds per unsaturated ester molecule. Alternatively, the unsaturated ester molecules have an average of at least 3 ester groups per unsaturated ester molecule and have an average of at least 1.5 carbon-carbon double bonds per unsaturated ester molecule. Alternatively, the unsaturated ester molecules have an average of at least 3 ester groups per unsaturated ester molecule and have an average of from 1.5 to 9 carbon-carbon double bonds per unsaturated ester molecule. As another alternative, the unsaturated ester molecules have an average of from 2 to 8 ester groups per unsaturated ester molecule and have an average of from 1.5 to 9 carbon-carbon double bonds per unsaturated ester oil.

In addition to the number (or average number) of ester groups and the number (or average number) of carbon-carbon double bonds present in the unsaturated ester molecules, the disposition of the carbon-carbon double bonds in unsaturated ester molecules having 2 or more carbon-carbon double bonds can be a consideration. In some embodiments where the unsaturated ester molecules have 2 or more carbon-carbon double bonds, the carbon-carbon double bonds can be conjugated. In other embodiments, the carbon-carbon double bonds can be separated from each other by only one carbon atom. When two carbon-carbon double bonds are separated by a carbon atom having two hydrogen atoms attached to it, e.g. a methylene group, these carbon-carbon double bonds can be termed as methylene interrupted double bonds. In yet other embodiments, the carbon-carbon double bonds are isolated, e.g. the carbon-carbon double bonds are separated from each other by 2 or more carbon atoms. In further embodiments, the carbon-carbon double bonds can be conjugated with a carbonyl group.

In some aspects, the unsaturated ester can be described as an ester of a polyol and unsaturated carboxylic acids. Within this description, the unsaturated carboxylic acid portion of the unsaturated ester can be called a polyol side chain (or more simply a side chain). In some embodiments, the unsaturated ester comprises less than 30 percent of side chains comprising methylene interrupted double bonds. In other embodiments, the unsaturated ester comprises greater than 30 percent of the side chains comprise methylene interrupted double bonds. In yet other embodiments, the unsaturated ester comprises less than 25 percent of side chains having 3 contiguous methylene interrupted carbon-carbon double bonds. In further embodiments, the unsaturated ester comprises less than 25 percent linolenic side chains. In further embodiments, the unsaturated ester comprises greater than 25 percent of side chains having 3 contiguous methylene interrupted carbon-carbon double bonds. In further embodiments, the unsaturated ester comprises greater than 25 percent linolenic side chains. In additional embodiments, the unsaturated ester comprises at least 30 percent side chains having 2 contiguous methylene interrupted carbon-carbon double bonds and less than 25 percent of side chains having 3 contiguous methylene interrupted carbon-carbon double bonds.

Additional functional groups can also be present in the unsaturated ester. A non-limiting list of functional groups include a hydroxy group, an ether group, an aldehyde group, a ketone group, an amine group, a carboxylic acid group among others, and combinations thereof. In an aspect, the unsaturated ester can comprise hydroxy groups. In some embodiments, the unsaturated esters have an average of at least 1.5 hydroxy groups per unsaturated ester molecule; alternatively, an average of at least 2 hydroxy groups per unsaturated ester molecule; alternatively, an average of at least 2.5 hydroxy groups per unsaturated ester molecule; or alternatively, an average of at least 3 hydroxy groups per unsaturated ester molecule. In other embodiments, the unsaturated esters have an average of from 1.5 to 9 hydroxy groups per unsaturated ester molecule; alternatively, an average of from 3 to 8 hydroxy groups per unsaturated ester molecule; alternatively, an average of from 2 to 4 hydroxy groups per unsaturated ester molecule; or alternatively, an average of from 4 to 8 hydroxy groups per unsaturated ester molecule. In an embodiment, the unsaturated ester comprises at least 2 hydroxy groups; alternatively, at least 3 hydroxy groups; or alternatively, at least 4 hydroxy groups. In other embodiments, the unsaturated ester comprises from 2 to 9 hydroxy groups; alternatively, from 2 to 4 hydroxy groups; alternatively, from 3 to 8 hydroxy groups; or alternatively, from 4 to 8 hydroxy groups.

The unsaturated ester utilized to produce the thiol ester utilized in aspects of this invention can be any unsaturated ester having the number of ester groups and carbon-carbon double bonds per unsaturated ester described herein. The unsaturated ester can be derived from natural sources, synthetically produced from natural source raw materials, produced from synthetic raw materials, produced from a mixture of natural and synthetic materials, or a combination thereof.

### Unsaturated Natural Source Oil

In an embodiment, the unsaturated ester is unsaturated natural source oil. The unsaturated natural source oil can be derived from naturally occurring nut, vegetable, plant, and animal sources. In an embodiment, the unsaturated ester comprises a triglyceride derived from naturally occurring nuts, vegetables, plant, and animal sources. In an embodiment, the unsaturated ester can be derived from genetically modified nuts, vegetables, plant, and animal sources. In an embodiment, the unsaturated ester oil comprises a triglyceride derived from genetically modified nuts, vegetables, plant, and animal sources.

In an aspect, the unsaturated natural source oil can be tallow, olive, peanut, castor bean, sunflower, sesame, poppy, seed, palm, almond seed, hazelnut, rapeseed, canola, soybean, corn, safflower, canola, cottonseed, camelina, flaxseed, or walnut oil. In some embodiment, the unsaturated natural source oil can be soybean, corn, castor bean, safflower, canola, cottonseed, camelina, flaxseed, or walnut oil. In further embodiments, the unsaturated natural source oil can be soybean oil; alternatively, corn oil; alternatively, castor bean oil; or alternatively, canola oil.

The unsaturated natural source oils are comprised of triglycerides that can be described as an ester of glycerol and an unsaturated carboxylic acid. Within this description, the unsaturated carboxylic acid portion of the triglyceride can be called a glycerol side chain (or more simply a side chain). In some embodiments, the triglyceride comprises less than 30 percent of side chains comprising methylene interrupted double bonds. In other embodiments, the triglyceride comprises greater than 30 percent of the side chains comprise methylene interrupted double bonds. In yet other embodiments, the triglyceride comprises less than 25 percent of side chains having 3 contiguous methylene interrupted carbon-carbon double bonds. In further embodiments, the triglyceride comprises less than 25 percent linolenic side chains. In further embodiments, the triglyceride comprises greater than 25 percent of side chains having 3 contiguous methylene interrupted carbon-carbon double bonds. In further embodiments, the triglyceride comprises greater than 25 percent linolenic side chains. In additional embodiments, the triglyceride comprises at least 30 percent side chains having 2 contiguous methylene interrupted carbon-carbon double bonds and less than 25 percent of side chains having 3 contiguous methylene interrupted carbon-carbon double bonds.

In another embodiment, the unsaturated natural ester oil comprises “natural” triglycerides derived from unsaturated natural source oils. In an embodiment, the unsaturated ester oil is synthetic. In an embodiment, the unsaturated ester oil comprises both synthetic and natural raw materials. In an embodiment, the unsaturated ester oil comprises synthetic triglycerides.

### Synthetic Unsaturated Esters

Synthetic unsaturated esters used as feedstock for aspects of this invention can be produced using methods for producing an ester group known to those skilled in the art. The term “ester group” means a moiety formed from the reaction of a hydroxy group and a carboxylic acid or a carboxylic acid derivative. Typically, the esters can be produced by reacting an alcohol with a carboxylic acid, transesterification of carboxylic acid ester with an alcohol, reacting an alcohol with a carboxylic acid anhydride, or reacting an alcohol with a carboxylic acid halide. Any of these methods can be used to produce the synthetic unsaturated ester oils used as a feedstock in an aspect of this invention. The alcohol, unsaturated carboxylic acid, unsaturated carboxylic acid ester, unsaturated carboxylic acid anhydride raw materials for the production of the unsaturated ester oil can be derived from natural, synthetic, genetic, or any combination of natural, genetic, and synthetic sources.

The polyols and the unsaturated carboxylic acids, simple unsaturated carboxylic acid esters, or unsaturated carboxylic acid anhydrides used to produce the unsaturated esters used as a feedstock in various aspects of this invention are independent elements. That is, these elements can be varied independently of each other and thus, can be used in any combination to produce an unsaturated ester utilized a feedstock to produce the compositions described in this application or as a feedstock for the processes described in this application.

### Synthetic Unsaturated Ester Oils—Polyol Component

The polyol used to produce the unsaturated ester oil can be any polyol or mixture of polyols capable of reacting with an unsaturated carboxylic acid, unsaturated simple carboxylic acid ester, carboxylic acid anhydride, or carboxylic acid halide under reaction conditions apparent to those skilled in the art.

The number of carbon atoms in the polyol can be varied. In one aspect, the polyol used to produce the unsaturated ester can comprise from 2 to 20 carbon atoms. In other embodiments, the polyol comprises from 2 to 10 carbon atoms; alternatively, from 2 to 7 carbon atoms; or alternatively, from 2 to 5 carbon atoms. In further embodiments, the polyol can be a mixture of polyols having an average of 2 to 20 carbon atoms; alternatively, an average of from 2 to 10 carbon atoms; alternatively, an average of 2 to 7 carbon atoms; or alternatively, an average of 2 to 5 carbon atoms.

In another aspect, the polyol used to produce the unsaturated ester can have any number of hydroxy groups needed to produce the unsaturated ester as described herein. In some embodiments, the polyol has 2 hydroxy groups; alternatively, 3 hydroxy groups; alternatively, 4 hydroxy groups; alternatively, 5 hydroxy groups; or alternatively, 6 hydroxy groups. In other embodiments, the polyol comprises at least 2 hydroxy groups; alternatively, at least 3 hydroxy groups; alternatively, at least 4 hydroxy groups; alternatively, at least 5 hydroxy groups; or alternatively, at least 6 hydroxy groups. In yet other embodiments, the polyol comprises from 2 to 8 hydroxy groups; alternatively, from 2 to 4 hydroxy groups; or alternatively, from 4 to 8 hydroxy groups.

In further aspects, the polyol used to produce the unsaturated ester is a mixture of polyols. In an embodiment, the mixture of polyols has an average of at least 1.5 hydroxy groups per polyol molecule. In other embodiments, the mixture of polyols has an average of at least 2 hydroxy groups per polyol molecule; alternatively, an average of at least 2.5 hydroxy groups per polyol molecule; alternatively, an average of at least 3.0 hydroxy groups per polyol molecule; or alternatively, an average of at least 4 hydroxy groups per polyol molecule. In yet another embodiments, the mixture of polyols has an average of 1.5 to 8 hydroxy groups per polyol molecule; alternatively, an average of 2 to 6 hydroxy groups per polyol molecule; alternatively, an average of 2.5 to 5 hydroxy groups per polyol molecule; alternatively, an average of 3 to 4 hydroxy groups per polyol molecule; alternatively, an average of 2.5 to 3.5 hydroxy groups per polyol molecule; or alternatively, an average of 2.5 to 4.5 hydroxy groups per polyol molecule.

In yet another aspect, the polyol or mixture of polyols used to produce the unsaturated thiol ester has a molecular weight or average molecular weight less than 1000. In other embodiments, the polyol or mixture of polyols have a molecular weight or average molecular weight less than 500; alternatively, less than 300; alternatively, less than 200; alternatively, less than 150; or alternatively, less than 100.

In some embodiments, suitable polyols include 1,2-ethanediol, 1,3-propanediol, 1,4-butanediol, 1,5-pentanediol, 1,6-hexanediol, dimethylolpropane, neopentyl glycol, 2-propyl-2-ethyl-1,3-propanediol, 1,2-propanediol, 1,3-butanediol, diethylene glycol, triethylene glycol, polyethylene glycol, dipropylene glycol, tripropylene glycol, polypropylene glycol, cyclohexanedimethanol, 1,3-dioxane-5,5-dimethanol, 1,4-xylylenedimethanol, 1-phenyl-1,2-ethanediol, trimethylolpropane, trimethylolethane, trimethylolbutane, glycerol, 1,2,5-hexanetriol, pentaerythritol, ditrimethylolpropane, diglycerol, ditrimethylolethane, 1,3,5-trihydroxybenzene, 1,4-xylylenedimethanol, 1-phenyl-1,2-ethanediol, or any combination thereof. In further embodiments, the polyol is glycerol, pentaerythritol, or mixtures thereof. In other embodiments, the polyol is glycerol; or alternatively, pentaerythritol. In an aspect, the polyol is castor oil.

### Synthetic Unsaturated Ester—Carboxylic Acid or Carboxylic Acid Equivalent Component

The carboxylic acid component of the unsaturated ester oil can be any carboxylic acid or mixture of carboxylic acids comprising a carbon-carbon double bond. As the carboxylic acid component will be combined with a polyol or polyol mixture comprising an average of greater than 1.5 hydroxy groups or any other number of hydroxy groups described herein, the carboxylic acid component can be any mixture comprising unsaturated carboxylic acids that produces an unsaturated ester oil meeting the feedstock requirement described herein. In some embodiments, the carboxylic acid component can be any mixture of saturated carboxylic acid and unsaturated carboxylic acid that produces an unsaturated ester oil meeting the feedstock requirement described herein. Thus, the carboxylic acid or carboxylic acid mixture used to produce the synthetic unsaturated ester oil can be described as having an average number of a specified element per carboxylic acid.

Independent elements of the carboxylic acid include the average number of carboxylic acid groups per carboxylic acid molecule, the average number of carbon atoms present in the carboxylic acid, and the average number of carbon-carbon double bonds per carboxylic acid. Additional independent elements include the position of the double bond in the carbon chain and the relative position of the double bonds with respect to each other when there are multiple double bonds.

Specific carboxylic acids used as a component of the carboxylic acid composition used to produce the unsaturated ester oil can have from 3 to 30 carbon atoms per carboxylic acid molecule. In some embodiments, the carboxylic acid is linear. In some embodiments, the carboxylic acid is branched. In some embodiments, the carboxylic acid is a mixture of linear and branched carboxylic acids. In some embodiments, the carboxylic acid can also comprise additional functional groups including alcohols, aldehydes, ketones, and epoxides, among others.

Suitable carboxylic acids that can be used as a component of unsaturated carboxylic acid composition can have from about 3 to about 30 carbon atoms; alternatively, 8 to 25 carbon atoms; or alternatively, from 12 to 20 carbon atoms. In other embodiments, the carboxylic acids comprising the unsaturated carboxylic acid composition comprise an average of 2 to 30 carbon atoms; alternatively, an average of 8 to 25 carbon atoms; or alternatively, an average of from 12 to 20 carbon atoms.

The carbon-carbon double bond can be located anywhere along the length of the carbon-carbon chain. In one embodiment, the double bond can be located at a terminal position. In another embodiment, the carbon-carbon double bond can be located at internal position. In yet another embodiment, the carboxylic acid or mixture of carboxylic acids can comprise both terminal and internal carbon-carbon double bonds. The double bond can also be described by indicating the number of substituents that are attached to the carbon-carbon double bond. In some embodiments, the carbon-carbon double bond can be mono-substituted, disubstituted, trisubstituted, tetrasubstituted, or a mixture of unsaturated carboxylic acids that can have any combination of monosubstituted, disubstituted, trisubstituted and tetrasubstituted carbon-carbon double bonds.

Suitable unsaturated carboxylic acids include acrylic, agonandoic, agonandric, alchornoic, ambrettolic, angelic, asclepic, auricolic, avenoleic, axillarenic, brassidic, caproleic, cetelaidic, cetoleic, civetic, coriolic, coronaric, crepenynic, densipolic, dihomolinoleic, dihomotaxoleic, dimorphecolic, elaidic, ephedrenic, erucic, gadelaidic, gadoleic, gaidic, gondolo, gondoleic, gorlic, helenynolic, hydrosorbic, isoricinoleic, keteleeronic, labellenic, lauroleic, lesquerolic, linelaidic, linderic, linoleic, lumequic, malvalic, mangold's acid, margarolic, megatomic, mikusch's acid, mycolipenic, myristelaidic, nervoic, obtusilic, oleic, palmitelaidic, petroselaidic, petroselinic, phlomic, physeteric, phytenoic, pyrulic, ricinelaidic, rumenic, selacholeic, sorbic, stearolic, sterculic, sterculynic, stillingic, strophanthus, tariric, taxoleic, traumatic, tsuduic, tsuzuic, undecylenic, vaccenic, vernolic, ximenic, ximenynic, ximenynolic, and combinations thereof. In further embodiments, suitable unsaturated carboxylic acids include oleic, palmitoleic, ricinoleic, linoleic, or any combinations thereof. Other suitable unsaturated carboxylic acids will be apparent to those persons having ordinary skill in the art and are to be considered within the scope of the present invention and combinations thereof.

In some embodiments, the unsaturated ester can be produced by transesterification of a simple ester of the carboxylic acid or mixture of carboxylic acids described herein with the polyol compositions described herein. In some embodiments, the simple ester is a methyl or ethyl ester of the carboxylic acid or mixture of carboxylic acids. In further embodiments, the simple carboxylic acid ester is a methyl ester of the carboxylic acids described herein.

### Epoxidized Unsaturated Esters

In an aspect, epoxidized unsaturated esters are used as a feedstock to produce materials described herein and for the processes to produce the material described herein. Generally, the epoxidized unsaturated ester can be derived by epoxidizing any unsaturated ester described herein. The unsaturated ester oil can be derived from natural sources, synthetically produced from natural source raw materials, produced from synthetic raw materials, produced from a mixture of natural and synthetic materials, or a combination thereof.

Minimally, the epoxidized unsaturated ester comprises at least one epoxide group. In an embodiment the epoxidized unsaturated ester comprises at least 2 epoxide groups; alternatively, at least 3 epoxide groups; or alternatively, at least 4 epoxide groups. In other embodiments, the epoxidized unsaturated ester comprises from 2 to 9 epoxide groups; alternatively, from 2 to 4 epoxide groups; alternatively, from 3 to 8 epoxide groups; or alternatively, from 4 to 8 epoxide groups.

In some embodiments, the unsaturated ester comprises a mixture of epoxidized unsaturated esters. In this aspect, the number of epoxide groups in the epoxidized unsaturated ester can be described as an average number of epoxide groups per epoxidized unsaturated ester molecule. In some embodiments, the epoxidized unsaturated esters have an average of at least 1.5 epoxide groups per epoxidized unsaturated ester molecule; alternatively, an average of at least 2 epoxide groups per epoxidized unsaturated ester molecule; alternatively, an average of at least 2.5 epoxide groups per epoxidized unsaturated ester molecule; or alternatively, an average of at least 3 epoxide groups per epoxidized unsaturated ester molecule. In other embodiments, the epoxidized unsaturated esters have an average of from 1.5 to 9 epoxide groups per epoxidized unsaturated ester molecule; alternatively, an average of from 3 to 8 epoxide groups per epoxidized unsaturated ester molecule; alternatively, an average of from 2 to 4 epoxide groups per epoxidized unsaturated ester molecule; or alternatively, an average of from 4 to 8 epoxide groups per epoxidized unsaturated ester molecule.

In an aspect the epoxidized unsaturated ester can be an epoxidized unsaturated natural source oil. The unsaturated natural source oil can be derived from naturally occurring nut, vegetable, plant, and animal sources. In an embodiment, the unsaturated ester oil is derived from genetically modified nuts, vegetables, plant, and animal sources. In an embodiment, the unsaturated ester oil comprises a triglyceride derived from genetically modified nuts, vegetables, plant, and animal sources.

In an aspect, the epoxidized natural source oil can be tallow, olive, peanut, castor bean, sunflower, sesame, poppy, seed, palm, almond seed, hazelnut, rapeseed, canola, soybean, corn, safflower, canola, cottonseed, camelina, flaxseed, or walnut oil. In some embodiment, the epoxidized natural source oil can be soybean, corn, castor bean, safflower, canola, cottonseed, camelina, flaxseed, or walnut oil. In further embodiments, the epoxidized natural source oil can be soybean oil; alternatively, corn oil; alternatively, castor bean oil; or alternatively, canola oil.

### Isocyanates

In an aspect, the thiourethane compositions of the present invention can be produced by contacting the thiol ester composition with the isocyanate composition. Generally, the isocyanate composition comprises an isocyanate having at least one isocyanate group. In embodiments, the isocyanate composition is comprised of molecules having multiple isocyanate groups. In some embodiments, the isocyanate composition comprises a mixture of isocyanate molecules. When the isocyanate composition comprises a mixture of isocyanate molecules, the isocyanate molecules can have an average of at least 1.5 isocyanate groups per isocyanate molecule; alternatively, an average of at least 2 isocyanate groups per isocyanate molecule; alternatively, an average of at least 2.5 isocyanate groups per isocyanate molecule; or alternatively, an average of at least 3 isocyanate groups per isocyanate molecule. In embodiments, the isocyanate molecules can have an average of from 1.5 to 12 isocyanate groups per isocyanate molecule; alternatively, an average of from 1.5 to 9 isocyanate groups per isocyanate molecule; alternatively, an average of from 2 to 7 isocyanate groups per isocyanate molecule; alternatively, an average of from 2 to 5 isocyanate groups per isocyanate molecule; or alternatively, an average of from 2 to 4 isocyanate groups per isocyanate molecule. In embodiments, the isocyanate composition can comprise aliphatic isocyanates, cycloaliphatic isocyanates, aromatic isocyanates, or any combination thereof. In some embodiments, the isocyanate composition comprises aliphatic isocyanates; alternatively, cycloaliphatic isocyanates; or alternatively, aromatic isocyanates.

In embodiments, the aliphatic isocyanates of the isocyanate composition can comprise ethylene diisocyanate, 1,3-trimethylene diisocyanate, 1,4-tetramethylene diisocyanate, 1,6-hexamethylene diisocyanate, 1,7-heptamethylene isocyanate, 1,8-octamethylene diisocyanate, 1,9-nonamethylene diisocyanate, 1,10-decamethylene diisocyanate, 1,11-undecamethylene diisocyanate, 1,12-dodeca-methylene diisocyanate, 2,2′-dimethylpentane diisocyanate, 2,2,4-trimethyl-1,6-hexamethylene diisocyanate, 2,4,4-trimethylhexamethylene diisocyanate, 1,6,11-undecane triisocyanate, 1,3,6-hexa-methylene triisocyanate, 1,8-diisocyanato-4-(isocyanatomethyl)octane, 2,5,7-trimethyl-1,8-diisocyanato-5-(isocyanatomethyl)octane, or any combination thereof. In some embodiments, the aliphatic isocyanates of the isocyanate composition can comprise ethylene diisocyanate, 1,3-trimethylene diisocyanate, 1,4-tetramethylene diisocyanate, 1,6-hexamethylene diisocyanate, 1,12-dodecamethylene diisocyanate, 2,2,4-trimethyl-1,6-hexamethylene diisocyanate, 2,4,4-trimethylhexamethylene diisocyanate, 1,6,11-undecane triisocyanate, 1,3,6-hexamethylene triisocyanate, or any combination thereof. In other embodiments, the aliphatic isocyanates of the isocyanate composition can comprise 1,4-tetramethylene diisocyanate, 1,6-hexamethylene diisocyanate, or any combination thereof. In yet other embodiments, the aliphatic isocyanate of the isocyanate composition comprises 1,6-hexamethylene diisocyanate.

In embodiments, the cycloaliphatic isocyanates of the isocyanate composition can comprise 1-iso-cyanato-2-isocyanatomethyl cyclopentane, 1,3-cyclohexane diisocyanate, 1,4-cyclohexane diisocyanate, 2,4-methylcyclohexane diisocyanate, 2,6-methylcyclohexane diisocyanate, 1,2-dimethylcyclohexane diisocyanate, 1,4-dimethylcyclohexane diisocyanate, isophorone diisocyanate (IPDI), 1-isocyanato-1-methyl-4(3)-isocyanatomethyl cyclohexane, 1,3-bis-(isocyanato-methyl) cyclohexane, 1,4-bis(isocyanato-methyl) cyclohexane, 2,4′-dicyclohexylmethane diisocyanate, 4,4′-dicyclohexylmethane diisocyanate (hydrogenated MDI, HMDI), 2,2′-dimethyldicyclohexylmethane diisocyanate, 4,4′-bis(3-methylcyclo-hexyl)methane diisocyanate, or any combination thereof. In some embodiments, the cyclic aliphatic isocyanates of the isocyanate composition can comprise 1,3-cyclohexane diisocyanate, 1,4-cyclohexane diisocyanate, 2,4-methylcyclohexane diisocyanate, 2,6-methylcyclohexane diisocyanate, 1,2-dimethyl-cyclohexane diisocyanate, 1,4-dimethylcyclohexane diisocyanate, isophorone diisocyanate, 2,4′-dicyclo-hexylmethane diisocyanate, 4,4′-dicyclohexylmethane diisocyanate, or any combination thereof. In other embodiments, the cyclic aliphatic isocyanate of the isocyanate composition comprises 1,3-cyclohexane diisocyanate; alternatively, 1,4-cyclohexane diisocyanate; alternatively, 2,4-methylcyclohexane diisocyanate; alternatively, 2,6-methylcyclohexane diisocyanate; alternatively, isophorone diisocyanate; alternatively, 2,4′-dicyclohexylmethane diisocyanate; or alternatively, 4,4′-dicyclohexylmethane diiso-cyanate.

In embodiments, the aromatic isocyanates of the isocyanate composition can comprise 1,3-phenylene diisocyanate, 1,4-phenylene diisocyanate, 2,4-tolylene diisocyanate (TDI), 2,5-toluene diisocyanate 2,6-tolylene diisocyanate, tolylene-α,4-diisocyante, 1,3-xylylene diisocyanate, 1,4-xylylene diisocyanate, diethylphenylene diisocyanate, diisopropylphenylene diisocyanate, trimethylbenzene triisocyanate, α,α,α′,α′-tetramethyl-1,3-xylylene diisocyanate, α,α,α′,α′-tetramethyl-1,4-xylylene diisocyanate, mesitylene triisocyanate, benzene triisocyanate, 1,5-diisocyanato naphthalene, methylnaphthalene diisocyanate, bis(isocyanatomethyl)naphthalene, biphenyl diisocyanate, 2,4′-diphenylmethane diisocyanate, 4,4′-diphenylmethane diisocyanate (MDI), polymeric 4,4′-diphenyl-methane diisocyanate (polymeric MDI, PMDI), 3,3′-dimethyl-diphenylmethane-4,4′-diisocyanate, bibenzyl-4,4′-diisocyanate, bis(isocyanatophenyl)ethylene, triphenylmethane triisocyanate, bis(isocyanatoethyl)benzene, bis-(isocyanatopropyl)benzene, bis(isocyanatobutyl)benzene, naphthalene triisocyanate, diphenylmethane-2,4,4′-triisocyanate, 3-methyldiphenylmethane-4,6,4′-triisocyanate, 4-methyldiphenyl-methane-3,5,2′,4′,6′-pentaisocyanate, tetrahydronaphthylene diisocyanate, or any combination thereof. In some embodiments, the aromatic isocyanates of the isocyanate composition can comprise 1,3-phenylene diisocyanate, 1,4-phenylene diisocyanate, 2,4-tolylene diisocyanate, 2,6-tolylene diisocyanate, 1,3-xylylene diisocyanate, 1,4-xylylene diisocyanate, trimethylbenzene triisocyanate, benzene triisocyanate, biphenyl diisocyanate, 2,4′-diphenylmethane diisocyanate, 4,4′-diphenylmethane diisocyanate, polymeric 4,4′-diphenylmethane diisocyanate, 3,3′-dimethyl-diphenylmethane-4,4′-diisocyanate, bibenzyl-4,4′-diisocyanate, triphenylmethane triisocyanate, polymeric MDI, naphthalene triisocyanate, or any combination thereof. In other embodiments, the aliphatic isocyanates of the isocyanate composition comprises 1,3-phenylene diisocyanate, 1,4-phenylene diisocyanate, 2,4-tolylene diisocyanate, 2,6-tolylene diisocyanate, 1,3-xylylene diisocyanate, 1,4-xylylene diisocyanate, 2,4′-diphenylmethane diisocyanate, 4,4′-diphenylmethane diisocyanate, polymeric 4,4′-diphenylmethane diisocyanate, 3,3′-dimethyl-diphenylmethane-4,4′-diisocyanate, polymeric 4,4′-diphenylmethane diisocyanate, or any combination thereof. In yet other embodiments, the aliphatic isocyanates of the isocyanate composition comprises 2,4-tolylene diisocyanate; alternatively, 2,6-tolylene diisocyanate; alternatively, 2,4- and 2,6-tolylene diisocyanate; alternatively, 4,4′-diphenylmethane diisocyanate; alternatively, polymeric 4,4′-diphenylmethane diisocyanate; or alternatively, mixtures of 2,4′-diphenylmethane diisocyanate and 4,4′-diphenylmethane diisocyanate.

### EXAMPLES

### Example 1—Preparation of Thiourethane Prepolymer Composition and Subsequent Formation of a Thiourethane Polymer

Samples were prepared of a mercaptanized soybean oil-based thiourethane prepolymer composition, which when cured can be useful as an adhesive. Table 1 provides the parameters that were used to prepare the thiourethane prepolymer samples and the adhesive properties of the moisture cured thiourethane polymer produced from the thiourethane prepolymers. 0.05 wt. % DBTDL (dibutyltin dilaurate) was used as the catalyst in some of the samples. In other samples. 0.5 wt. % DMDEE (dimorpholinodiethylether) was used as the catalyst. Once the thiourethane prepolymer compositions were prepared, the response variables for the thiourethane prepolymers were observed (initial and 5 day apparent viscosity). The thiourethane prepolymers were then moisture cured and the adhesion of the resulting thiourethane polymer composition to various substrates was measured. When measuring the adhesion of the cured thiourethane polymer composition, a scale of 1-5 was used. In this scale, 5=excellent, 4=very good, 3=good, 2=fair, and 1=poor. The results of the observations of the response variable are described in Example 2.

TABLE 1

| | Control Variables | | Response Variables | |
|---|---|---|---|---|
| Constants | Variable | Levels | Response | Units |
| MSO functionality ~2.6 [8.6 wt. | wt. % PPG in mixture | 10% | Thiourethane | cP (@ 65° C.) |
| % mercaptan sulfur | | 20% | Prepolymer Initial | |
| | | 30% | Apparent Viscosity | |
| | | 40% | | |
| Diol oligomer type = polypropylene | NCO Index (mol. ratio | 3.5 | Thiourethane | cP (@ 65° C.) |
| glycol (PPG), | of NCO:XH groups) | 3.0 | Prepolymer 5 Day | |
| Mn ~1000 | | 2.5 | Apparent Viscosity | |
| | | 2.0 | | |
| Type of isocyanate = MDI | Catalyst type | DBTDL, | Cured Thiourethane | 1–5 |
| unmodified, E.W. = 125.125 | | DMDEE | Polymer Adhesion to | |
| | | | various substrates | |
| | | | (wood, CRS, A1, | |
| | | | concrete) | |

Reaction Conditions = 80° C. 30 min., 80° C. under vacuum 30 min.

### Example 2—Observations of Response Variable from Example 1

Eight different 350 gram thiourethane prepolymer composition samples were prepared using mercaptanized soybean oil, MDI, and polypropylene glycol having a Mn≈1000 in accordance with the parameters shown in Table 1. Each thiourethane prepolymer composition sample was applied to a 4″×4″ plate and was allowed to cure overnight (by moisture cure). The relative adhesion of each thiourethane polymer composition to the substrate upon which it was applied was evaluated to determine how well the material adhered to the substrate in accordance with ASTM Standard D3808. Each prepolymer composition was applied to the desired substrate from a 60 cc syringe in a drop size of approximately 2 cm in diameter. The substrates included concrete cinderblock, cold-rolled steel Q-Panel® plates, aluminum Q-Panel® plates, and oak planks. The prepolymer compositions were allowed to cure (to produce a thiourethane polymer composition) at room conditions for 24 hours, after which the cured droplets were pried using a thin-bladed stainless steel spatula to examine the relative ease of separation and the failure type of each cured prepolymer composition from the substrates. A relative adhesion scale of 1-5 was used in comparing cured prepolymer compositions, with 5=excellent, 4=very good, 3=good, 2=fair, and 1=poor. The results are shown in Table 2.

TABLE 2

| | Sample No. | | | | | | | |
|---|---|---|---|---|---|---|---|---|
| | 1 | 2 | 3 | 4 | 5 | 6 | 7 | 8 |
| MSO (g) | 131.1 | 130.5 | 114.0 | 113.5 | 98.4 | 98.0 | 84.9 | 84.5 |
| MSO wt. % | 37.5 | 37.3 | 32.6 | 32.4 | 28.1 | 28.0 | 24.2 | 24.1 |
| Olig. Wt. % | 10 | 10 | 20 | 20 | 30 | 30 | 40 | 40 |
| Olig. Wt. (g) | 35.0 | 34.8 | 70.0 | 69.7 | 104.9 | 104.5 | 139.9 | 139.3 |
| MDI (g) | 183.8 | 182.9 | 165.9 | 165.1 | 146.4 | 145.8 | 125.0 | 124.5 |
| NCO/SH + OH mol | 3.5 | 3.5 | 3.0 | 3.0 | 2.5 | 2.5 | 2.0 | 2.0 |
| ratio | | | | | | | | |
| OH:SH mol ratio | 0.2 | 0.2 | 0.4 | 0.4 | 0.8 | 0.8 | 1.2 | 1.2 |
| Cat. | DBTDL | DMDEE | DBTDL | DMDEE | DBTDL | DMDEE | DBTDL | DMDEE |
| Cat. Wt. (g) | 0.175 | 1.750 | 0.175 | 1.750 | 0.175 | 1.750 | 0.175 | 1.750 |
| Cat. Level (wt. %) | 0.05 | 0.50 | 0.05 | 0.50 | 0.05 | 0.50 | 0.05 | 0.50 |
| Wt. % NCO | 11.8 | 10.1 | 10.3 | 9.3 | 7.8 | 7.5 | 6.0 | 5.3 |
| Theoretical % | 12.6 | 12.5 | 10.6 | 10.6 | 8.4 | 8.4 | 6.0 | 6.0 |
| NCO | | | | | | | | |
| Initial Visc. (cP) | 3400 | 2793 | 3840 | 4495 | 7650 | 6570 | 8860 | 7600 |
| Relative Adhesion | 5 | 5 | 5 | 5 | 5 | 5 | 5 | 5 |
| to Wood | | | | | | | | |
| Relative Adhesion | 4 | 4 | 4 | 4 | 5 | 5 | 5 | 5 |
| to CRS | | | | | | | | |
| Relative Adhesion | 4 | 4 | 4 | 4 | 5 | 4 | 5 | 4 |
| to Aluminum | | | | | | | | |
| Relative Adhesion | 4 | 4 | 4 | 4 | 5 | 5 | 5 | 5 |
| to Concrete | | | | | | | | |

### Example 3—Preparation of Thiourethane Polymer Compositions Prepared from 2.0 Isocyanate Index Thiourethane Prepolymer Compositions

Samples of thiourethane polymers were prepared using the thiourethane prepolymer compositions having a 2.0 isocyanate index using the constants and control variables contained in Table 3. The response variables of the thiourethane polymer compositions were observed when the control variables of the thiourethane prepolymer were changed. The results of the observations are described in Example 4.

TABLE 3

| | | | Thiourethane Polymer | |
|---|---|---|---|---|
| | Control Variables | | Response Variables | |
| Constants | Variable | Levels | Response | Units |
| Mold - Teflon | Sealant Type | MSOPPG-DBTDL - 2.0 | Shore Hardness A | # |
| molds | | MSOPPG-DMDEE - 2.0 | (ASTM D 2240) | |
| | | Bostik ® GPS 1 | | |
| | | Bostik ® 915 | | |
| Cure Profile - R.T., | | | Tensile modulus @ 25% | psi |
| 50% RH, 14 days | | | % elongation (ASTM | |
| | | | D412) | |
| | | | Tensile modulus @ 50% | psi |
| | | | elongation (ASTM | |
| | | | D412) | |
| | | | Tensile modulus @ | psi |
| | | | break (ASTM D412) | |
| | | | Elongation @ break | % |
| | | | (ASTM D412) | |

### Example 4—Observations of Response Variables from Example 3

Thiourethane polymer samples were prepared using a 2.0 isocyanate index (ratio of NCO:SH+OH groups and a OH:SH molar ratio of 1.2:1) thiourethane prepolymer in accordance with the variables listed in Table 3 and were compared with samples prepared using commercially available sealants from Bostik. The shore hardness A, modulus at 25% elongation, modulus at 50% elongation, tensile strength, and elongation of the thiourethane polymer samples prepared in accordance with embodiments of the present invention and of the control samples prepared using Bostik® adhesives are located in Table 4.

TABLE 4

| | | | | | Tensile | |
|---|---|---|---|---|---|---|
| | | Shore | Modulus @ | Modulus @ | Strength | Elongation |
| Run | Sealant Type | Hardness A | 25% (psi) | 50% (psi) | (psi) | (%) |
| 1 | MSO-PPG-DBTDL-2.0 | 47.0 | 760 | 580 | 383.8 | 75.9 |
| 2 | MSO-PPG-DBTDL-2.0 | 51.3 | 1160 | 840 | 697.7 | 96.1 |
| 3 | MSO-PPG-DBTDL-2.0 | 43.7 | 840 | 600 | 528.0 | 93.3 |
| 4 | MSO-PPG-DBTDL-2.0 | 58.7 | 780 | 570 | 287.8 | 50.8 |
| 5 | MSO-PPG-DBTDL-2.0 | 58.3 | 740 | 540 | 419.0 | 84.7 |
| 6 | MSO-PPG-DMDEE-2.0 | 30.0 | 480 | 390 | 294.8 | 85.3 |
| 7 | MSO-PPG-DMDEE-2.0 | 27.0 | 396 | 310 | 227.6 | 85.7 |
| 8 | MSO-PPG-DMDEE-2.0 | 31.7 | 352 | 290 | 204.7 | 87.3 |
| 9 | MSO-PPG-DMDEE-2.0 | 36.0 | 392 | 320 | 209.3 | 73.5 |
| 10 | MSO-PPG-DMDEE-2.0 | 44.0 | 460 | 370 | 226.5 | 64.7 |
| 11 | Bostik ® GPS1 | 25.8 | 168 | 120 | 89.6 | 437.6 |
| 12 | Bostik ® GPS1 | 22.2 | 160 | 98 | 74.0 | 422.8 |
| 13 | Bostik ® GPS1 | 28.1 | 188 | 112 | 84.4 | 423.8 |
| 14 | Bostik ® GPS1 | 29.7 | 168 | 102 | 76.8 | 410.0 |
| 15 | Bostik ® GPS1 | 25.7 | 156 | 98 | 73.1 | 423.2 |
| 16 | Bostik ® 915 | 15.8 | 112 | 84 | 69.9 | 413.0 |
| 17 | Bostik ® 915 | 19.8 | 108 | 84 | 66.2 | 387.9 |
| 18 | Bostik ® 915 | 21.1 | 112 | 82 | 72.7 | 408.2 |
| 19 | Bostik ® 915 | 20.0 | 112 | 80 | 68.3 | 435.5 |

### Example 5—Varying Isocyanate Index Thiourethane Prepolymer Compositions

Several thiourethane prepolymer compositions were prepared having an isocyanate index that ranged from 4.0 to 6.0. The initial apparent viscosity and the apparent viscosity after five days were measured, the results of which are shown in Table 5. In runs 1, 2, 3, and 8, the initial and five day sample viscosities were measured at 100° F. (38° C.). In runs 4, 6, 9, 13, and 17, the initial and five day sample viscosities were measured at 120° F. (49° C.). In run 5, the initial apparent viscosity was measured at 100° F. (38° C.) and the five day apparent viscosity was measured at 120° F. (49° C.). In run 7, the initial apparent viscosity was measured at 120° F. (49° C.) and the five day apparent viscosity was measured at 140° F. (60° C.). In run 16, the initial and five day sample viscosities were measured at 140° F. (60° C.).

TABLE 5

| | | | | | | Cat. | | | Initial | 5 Day |
|---|---|---|---|---|---|---|---|---|---|---|
| | MSO | MDI | NCO/SH | | Cat. | Level | Wt. % | Theor. % | Visc. | Visc. |
| Run | (g) | (g) | mol ratio | Catalyst | wt. (g) | (wt. %) | NCO | NCO | (cP) | (cP) |
| 1 | 117.0 | 232.8 | 6.0 | DMDEE | 0.175 | 0.05% | 18.3% | 18.6% | 664 | 2049 |
| 2 | 116.5 | 231.8 | 6.0 | DMDEE | 1.750 | 0.50% | 17.4% | 18.5% | 1984 | 2742 |
| 3 | 131.3 | 217.7 | 5.0 | DBTDL | 0.980 | 0.28% | 15.8% | 16.7% | 6360 | 9050 |
| 4 | 150.3 | 199.5 | 4.0 | DBTDL | 0.175 | 0.05% | 13.8% | 14.4% | 6240 | 6280 |
| 5 | 131.0 | 217.3 | 5.0 | DMDEE | 1.750 | 0.50% | 15.6% | 16.7% | 7600 | 4080 |
| 6 | 131.6 | 218.2 | 5.0 | DMDEE | 0.175 | 0.05% | 16.1% | 16.8% | 1143 | 1405 |
| 7 | 150.0 | 199.0 | 4.0 | DBTDL | 0.980 | 0.28% | 13.6% | 14.3% | 8990 | 7920 |
| 8 | 117.0 | 232.8 | 6.0 | DBTDL | 0.175 | 0.05% | 17.2% | 18.6% | 2445 | 1716 |
| 9 | 116.5 | 231.8 | 6.0 | DBTDL | 1.750 | 0.50% | 17.0% | 18.5% | 1860 | 4065 |
| 10 | 149.7 | 198.6 | 4.0 | DMDEE | 1.750 | 0.50% | 13.2% | 14.3% | 8480 | 7940 |
| 11 | 150.0 | 199.0 | 4.0 | DMDEE | 0.980 | 0.28% | | 14.3% | | |
| 12 | 131.6 | 218.2 | 5.0 | DBTDL | 0.175 | 0.05% | | 16.8% | | |
| 13 | 150.3 | 199.5 | 4.0 | DMDEE | 0.175 | 0.05% | 13.7% | 14.4% | 4000 | 4760 |
| 14 | 116.7 | 232.3 | 6.0 | DMDEE | 0.980 | 0.28% | | 18.6% | | |
| 15 | 116.7 | 232.3 | 6.0 | DBTDL | 0.980 | 0.28% | | 18.6% | | |
| 16 | 149.7 | 198.6 | 4.0 | DBTDL | 1.750 | 0.50% | 13.4% | 14.3% | 7150 | >10,000* |
| 17 | 131.3 | 217.7 | 5.0 | DMDEE | 0.980 | 0.28% | 15.6% | 16.7% | 1816 | 1972 |
| 18 | 131.0 | 217.3 | 5.0 | DBTDL | 1.750 | 0.50% | | 16.7% | | |

*The reading exceeded the upper limit of the Brookfield ® viscometer.

### Example 6—Thiourethane Prepolymer Compositions Having a 4.0 Isocyanate Index

Six thiourethane prepolymer compositions samples were prepared using an isocyanate index of 4.0. The initial apparent viscosity of the thiourethane prepolymer samples was measured and is shown in Table 6.

TABLE 6

| | | | | | | | | Initial |
|---|---|---|---|---|---|---|---|---|
| | | | | | Cat. | | | Apparent |
| | | | | Cat. Wt. | Level | wt. % | Theoretical | Viscosity |
| Run | MSO (g) | MDI (g) | Catalyst | (g) | (wt. %) | NCO | % NCO | (cP) |
| 1 | 298.7 | 401.0 | DBTDL | 0.35 | 0.05% | 13.7% | 14.4% | 1816 |
| 2 | 298.7 | 401.0 | DBTDL | 0.35 | 0.05% | 13.7% | 14.4% | 1846 |
| 3 | 298.7 | 401.0 | DBTDL | 0.35 | 0.05% | 13.6% | 14.4% | 2463 |
| 4 | 297.3 | 399.2 | DMDEE | 3.50 | 0.50% | 13.5% | 14.4% | 2808 |
| 5 | 297.3 | 399.2 | DMDEE | 3.50 | 0.50% | 13.5% | 14.4% | 2901 |
| 6 | 297.3 | 399.2 | DMDEE | 3.50 | 0.50% | 13.4% | 14.4% | 2943 |

### APPLICATIONS

In addition to the uses described herein, embodiments of the present invention are useful in other numerous applications. For example, embodiments of the invention are useful in various polymer applications that include, but are not limited to, as thiourethanes, foams, adhesives, and sealants. It is believed that the compositions described herein can also be used as epoxy hardening agents, polyacrylates and polymethacrylate templates for paints and polyester resins, printing ink binder polymers, alkyd resin cross-linkers, sulfur based paint template, radiation cured polymers, mining and drilling chemicals, specialty chain transfer agents, rubber modifiers, and the like. Because the feedstock materials are economical and readily available, it is believed that embodiments of the present would be useful in such applications and others.

While this invention has been described with reference to illustrative embodiments and examples, the description is not intended to be construed in a limiting sense. Thus, various modifications of the illustrative embodiments, as well as other embodiments of the invention, will be apparent to persons skilled in the art upon reference to this description. It is therefore contemplated that the appended claims will cover any such modifications or embodiments.

All publications, patents and patent applications referred to herein are incorporated by reference in their entirety to the same extent as if each individual publication, patent or patent application was specifically and individually indicated to be incorporated by reference in its entirety.

What is claimed is:

1. A thiourethane prepolymer composition comprising a reaction product of a mixture comprising:
a. a thiol ester composition comprising an average of at least two thiol groups per thiol ester molecule;
b. an isocyanate composition comprising an average of at least two isocyanate groups per isocyanate molecule; and
c. a catalyst;
wherein the thiol ester composition and the isocyanate composition have a NCO:XH equivalent ratio of greater than 2.1:1, and wherein NCO represents the isocyanate groups used to produce the thiourethane prepolymer composition and XH represents active hydrogen groups used to produce the thiourethane prepolymer composition, the active hydrogen groups being selected from the group consisting of the thiol groups, alcohol groups, amine groups, amide groups, carboxylic acid groups, and combinations thereof.

2. The composition of claim 1, wherein the thiol ester composition comprises a mercaptanized unsaturated natural source oil, a mercaptanized epoxidized natural source oil, a crosslinked mercaptanized natural source oil, or combinations thereof.

3. The composition of claim 1, wherein the thiol ester composition comprises a mercaptanized soybean oil, a mercaptanized epoxidized soybean oil, a crosslinked mercaptanized soybean oil, or combinations thereof.

4. The composition of claim 1, wherein the catalyst comprises a tin catalyst, an amine catalyst, a bismuth catalyst, an iron catalyst, or combinations thereof.

5. The composition of claim 1, further comprising a solvent.

6. The composition of claim 5, wherein the solvent is methyl ethyl ketone, glycerol carbonate, acetone, hexene, petroleum distillate, butyl acetate, toluene, benzene, or combinations thereof.

7. The composition of claim 1, further comprising a property modifying agent.

8. The composition of claim 7, wherein the property modifying agent comprises a polypropylene glycol.

9. The composition of claim 1, wherein the NCO:XH equivalent ratio ranges from 2.1:1 to 10:1.

10. The composition of claim 1 having an apparent viscosity that ranges from 2500 centipoises to 10000 centipoises at a temperature of 120° F. (49° C.).

11. A thiourethane polymer composition comprising a cured reaction product of a mixture comprising a thiol ester composition, an isocyanate composition, and a catalyst, the thiourethane polymer composition having a full cure lap shear strength on oak-to-oak substrate per ASTM D1002 in a range of 300 psi (2068 kPa) to 1500 psi (10340 kPa).

12. The thiourethane polymer composition of claim 11, wherein the thiol ester composition comprises a mercaptanized unsaturated natural source oil, a mercaptanized epoxidized natural source oil, a crosslinked mercaptanized natural source oil, or combinations thereof.

13. The thiourethane polymer composition of claim 11, wherein the thiol ester composition comprises a mercaptanized soybean oil, a mercaptanized epoxidized soybean oil, a crosslinked mercaptanized soybean oil, or combinations thereof.

14. The thiourethane polymer composition of claim 11 having an average elongation value that ranges from 50% to 100%.

15. The thiourethane polymer composition of claim 11 having a joint mobility test result that ranges between −45% and 45%.

16. The thiourethane polymer composition of claim 11 having an average shore hardness A value ranging from 25 to 60.

17. The thiourethane polymer composition of claim 11 having an average modulus at 25% elongation ranging from 350 psi (2413 kPa) to 1175 psi (8101 kPa).

18. The thiourethane polymer composition of claim 11 having an average modulus at 50% elongation ranging from 250 psi (1724 kPa) to 850 psi (5861 kPa).

19. The thiourethane polymer composition of claim 11 having an average tensile strength ranging from 200 psi (1379 kPa) to 700 psi (4826 kPa).

20. A method of preparing a thiourethane polymer composition comprising the steps of:
a. contacting a thiol ester composition comprising an average of at least two thiol groups per thioester molecule, an isocyanate composition comprising an average of at least two isocyanate groups per isocyanate molecule, and a catalyst to form a thiourethane prepolymer composition, the thiol ester composition and the isocyanate composition having a NCO:XH equivalent ratio greater than 2.1:1, wherein NCO represents the isocyanate groups used to produce the thiourethane prepolymer composition and XH represents active hydrogen groups used to produce the thiourethane prepolymer composition, the active hydrogen groups being selected from the group consisting of the thiol groups, alcohol groups, amine groups, amide groups, carboxylic acid groups, and combinations thereof; and
b. curing the thiourethane prepolymer composition to produce the thiourethane polymer composition having a full cure lap shear strength on oak-to-oak substrate per ASTM D1002 in a range of 300 psi (2068 kPa) to 1500 psi (10340 kPa).

21. The method of claim 20, wherein the thiol ester composition comprises a mercaptanized unsaturated natural source oil, a mercaptanized epoxidized natural source oil, a crosslinked mercaptanized natural source oil, or combinations thereof.

22. The method of claim 20, wherein the thiol ester composition comprises a mercaptanized soybean oil, a mercaptanized epoxidized soybean oil, a crosslinked mercaptanized soybean oil, or combinations thereof.

23. The method of claim 20, wherein the catalyst comprises a tin catalyst, an amine catalyst, a bismuth catalyst, an iron catalyst, or combinations thereof.

24. The method of claim 20, wherein the NCO:XH equivalent ratio ranges from 2.1:1 to 10:1.

25. The method of claim 20, wherein contacting the thiol ester composition, the isocyanate composition, and the catalyst further comprises contacting a property modifying agent.

26. The method of claim 25, wherein the property modifying agent comprises a polypropylene glycol.

27. The method of claim 20, wherein contacting the thiol ester composition, the isocyanate composition and the catalyst further comprises contacting a solvent.

28. The method of claim 27, wherein the solvent is methyl ethyl ketone, glycerol carbonate, acetone, hexene, petroleum distillate, butyl acetate, toluene, benzene, or combinations thereof.

29. The method of claim 20, wherein the thiourethane polymer composition has an average elongation value that ranges from 50% to 100%.

30. The method of claim 20, wherein the thiourethane polymer composition has a joint mobility test result that ranges between −45% and 45%.

31. The method of claim 20, wherein the thiourethane polymer composition has an average shore hardness A value ranging from 25 to 60.

32. The method of claim 20, wherein the thiourethane polymer composition has an average modulus at 25% elongation ranging from 350 psi (2413 kPa) to 1175 psi (8101 kPa).

33. The method of claim 20, wherein the thiourethane polymer composition has an average modulus at 50% elongation ranging from 250 psi (1724 kPa) to 850 psi (5861 kPa).

34. The method of claim 20, wherein the thiourethane polymer composition has an average tensile strength ranging from 200 psi (1379 kPa) to 700 psi (4826 kPa).

35. The method of claim 20, wherein curing the thiourethane prepolymer composition includes using a multi-component cure system wherein the thiourethane prepolymer composition is contacted with an active hydrogen agent and optionally a second catalyst.
